SEQUENCE LISTING (11 sequences)

SEQ ID NO: 1 - DNA, 20 bp, synthetic construct
  source 1..20: mol_type = other DNA
gaccccaaaatcagcgaaat

SEQ ID NO: 2 - DNA, 24 bp, synthetic construct
  source 1..24: mol_type = other DNA
tctggttactgccagttgaatctg

SEQ ID NO: 3 - DNA, 12 bp, synthetic construct
  source 1..12: mol_type = other DNA
  modified_base 1: mod_base = OTHER; note = Fluorescein-accccgcat-ZEN quencher modified nucleotide
  modified_base 12: mod_base = OTHER; note = 3' Iowa Black FQ (IDT) quencher modified nucleotide
tacgtttggacc

SEQ ID NO: 4 - DNA, 19 bp, synthetic construct
  source 1..19: mol_type = other DNA
agatttggacctgcgagcg

SEQ ID NO: 5 - DNA, 20 bp, synthetic construct
  source 1..20: mol_type = other DNA
gagcggctgtctccacaagt

SEQ ID NO: 6 - DNA, 14 bp, synthetic construct
  source 1..14: mol_type = other DNA
  modified_base 1: mod_base = OTHER; note = Hexachloro-Fluorescein-ttctgacct-ZEN quencher modified nucleotide
  modified_base 14: mod_base = OTHER; note = 3' Iowa Black FQ (IDT) quencher modified nucleotide
gaaggctctgcgcg

SEQ ID NO: 7 - DNA, 24 bp, synthetic construct
  source 1..24: mol_type = other DNA
  modified_base 1: mod_base = OTHER; note = Fluorescein modified nucleotide
  modified_base 24: mod_base = OTHER; note = Black Hole Quencher-1 modified nucleotide
accccgcattacgtttggtggacc

SEQ ID NO: 8 - DNA, 20 bp, synthetic construct
  source 1..20: mol_type = other DNA
ttacaaacattggccgcaaa

SEQ ID NO: 9 - DNA, 18 bp, synthetic construct
  source 1..18: mol_type = other DNA
gcgcgacattccgaagaa

SEQ ID NO: 10 - DNA, 23 bp, synthetic construct
  source 1..23: mol_type = other DNA
  modified_base 1: mod_base = OTHER; note = SUN flurophore modified nucelotide
  modified_base 23: mod_base = OTHER; note = Black Hole Quencher-1 modified nucleotide
acaatttgcccccagcgcttcag

SEQ ID NO: 11 - DNA, 23 bp, synthetic construct
  source 1..23: mol_type = other DNA
  modified_base 1: mod_base = OTHER; note = 5' carboxy-X-rhodamine modified nucleotide
  modified_base 23: mod_base = OTHER; note = Black Hole Quencher-1 modified nucleotide
ttctgacctgaaggctctgcgcg

(19) United States
(12) Patent Application Publication
(10) Pub. No.: US 2023/0203566 A1
(43) Pub. Date: Jun. 29, 2023

(54) Apparatus and Methods for Rapid Nucleic Acid Detection

(71) Applicants: The Trustees of Columbia University in the City of New York, New York, NY (US); Rover Diagnostics, Inc., Greenvale, NY (US)

(72) Inventors: Samuel K. SIA, New York, NY (US); Nicole R. BLUMENFELD, New York, NY (US); Mark FASCIANO, Port Washington, NY (US); Bulent ONALIR, Englewood, NJ (US); Martin JASPAN, Somerville, MA (US)

(73) Assignees: The Trustees of Columbia University in the City of New York, New York, NY (US); Rover Diagnostics, Inc., Greenvale, NY (US)

(21) Appl. No.: 17/986,121

(22) Filed: Nov. 14, 2022

Related U.S. Application Data
(63) Continuation of application No. PCT/US2021/032404, filed on May 14, 2021.
(60) Provisional application No. 63/025,420, filed on May 15, 2020.
(60) Provisional application No. 63/086,956, filed on Oct. 2, 2020.

(51) Int. Cl. C12Q 1/6816; C12Q 1/6876; C12Q 1/686

(52) U.S. Cl. CPC C12Q 1/6816; C12Q 1/6876; C12Q 1/686; C12Q 2600/16

(57) ABSTRACT

Methods and apparatus for rapid and accurate detection of nucleic acid in a single reaction chamber are provided. In one aspect, a patient specimen suspected of comprising a first nucleic acid is used to form a crude lysate which is combined with an infrared absorbing material, a detecting nucleic acid, and at least one reporter molecule in the single reaction chamber and heated by irradiating the reaction mixture with infrared light. Another aspect is directed to an apparatus for detecting a presence or absence of a plurality of different molecules within a reaction container. The apparatus comprises an infrared light source aimed to illuminate contents of the reaction container; an excitation light source positioned to illuminate contents of the reaction container; and a spectrometer positioned to detect emission light emanating from the reaction container.

### CROSS REFERENCE TO RELATED APPLICATIONS

This Application is a continuation of International Application PCT/US2021/032404, filed May 14, 2021, which claims the benefit of U.S. Provisional Application 63/025,420, filed May 15, 2020, and US Provisional Application 63/086,956, filed Oct. 2, 2020, each of which is incorporated herein by reference in its entirety. All references cited herein, including but not limited to patents and patent applications, are incorporated by reference in their entirety.

### STATEMENT REGARDING FEDERALLY-SPONSORED RESEARCH

This invention was made with government support under grant HL143541 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Mar. 15, 2023, is named 1448-0221US01 SL.xml and is 16,449 bytes in size.

### BACKGROUND

In Dec. 2019, a previously unknown betacoronavirus was identified in Wuhan, China and discovered to cause, sometimes life-threatening, pneumonia-like symptoms. Over the next few months, the novel coronavirus, named SARS-CoV-2, continued to spread around the world, sickening millions of people with COVID-19 and causing hundreds of thousands of deaths.

Since then, the world has continued to look for improved methods to contain the spread of the virus. Although there is universal agreement on the critical value of widespread, distributed molecular testing for SARS-CoV-2 in the global response to the COVID-19 pandemic, such testing is not currently achievable using complicated and expensive molecular technologies. The current FDA EUA-approved point-of-care COVID-19 molecular tests are either too expensive and insensitive or too slow and complex for large-scale use. There is an urgent need for an affordable, fast, and simple-to-use molecular test, to ascertain the infection status of up to tens of millions of people per week, in a manner that avoids chaotic and socially dense queues at limited testing sites.

What is needed is a highly sensitive and reliable gold standard test that can be made available in an affordable and rapid manner to locations around the country and world, and which could be developed and offered in the near future. Such a test would be useful for a large number of viruses (including SARS-CoV-2, flu, and others), bacteria, and infectious-disease targets.

### SUMMARY

Aspects described herein provide a first method of detecting a nucleic acid in a single reaction chamber by (a) obtaining a patient specimen suspected of comprising a first nucleic acid, (b) forming a crude lysate from the patient specimen, (c) forming a reaction mixture by combining the crude lysate with infrared absorbing materials, a detecting nucleic acid, and at least one reporter molecule in the single reaction chamber, (d) heating the reaction mixture to at least 35° C. by irradiating the reaction mixture with infrared light, and (e) detecting a presence of the at least one reporter molecule, wherein a presence of the at least one reporter molecule indicates the patient specimen contains the first nucleic acid, and wherein steps (b) through (d) occur in the single reaction chamber.

Aspects described herein provide a second method for detecting a presence or absence of a plurality of different molecules within a reaction container comprising (a) illuminating contents of the reaction container using infrared light until a temperature within the reaction container reaches a denaturing temperature; (b) allowing the heated contents of the reaction container to cool until a temperature within the reaction container reaches an annealing temperature; (c) illuminating the contents of the reaction container with excitation light; (d) obtaining, while the contents of the reaction container are being illuminated with the excitation light, a respective measured spectrum of light that is being emitted by the contents of the reaction container; (e) deconvolving the respective measured spectrum into a plurality of respective individual spectra, each of which corresponds to a respective one of the different molecules; and (f) outputting data corresponding to each of the respective individual spectra. Steps (a) through (f) are repeated at least 10 times.

Aspects described herein provide a first apparatus for detecting a presence or absence of a plurality of different molecules within a reaction container. The first apparatus comprises an infrared light source aimed to illuminate contents of the reaction container; an excitation light source positioned to illuminate contents of the reaction container; and a spectrometer positioned to detect emission light emanating from the reaction container during times when the excitation light source is illuminating the contents of the reaction container. The first apparatus also comprises a controller programmed to, for each of N cycles, (a) control the infrared light source so that the temperature within the reaction container cycles between a denaturing temperature and an annealing temperature, (b) obtain, from the spectrometer, a respective measured spectrum in response to the excitation light, (c) deconvolve the respective measured spectrum into a plurality of respective individual spectra, each of which corresponds to a respective one of the different molecules, and (d) output data corresponding to each of the respective individual spectra, wherein N is an integer greater than or equal to 10.

Further aspects provide a second apparatus for detecting the presence or absence of a nucleic acid within a reaction container. The reaction container has a longitudinal axis. The second apparatus has a support structure shaped and dimensioned to hold the reaction container so that contents of the reaction container occupy a region in space; an infrared light source aimed in a first direction so as to illuminate at least a first portion of the region in space; an infrared thermometer aimed in a second direction so as to obtain temperature readings from at least a second portion of the region in space; a first-wavelength light source aimed in a third direction so as to illuminate at least a third portion of the region in space; and a light detector aimed in a fourth direction, wherein the light detector detects second-wavelength light arriving from the third portion of the region in space. The first and second wavelengths are selected such that when reporter molecules are illuminated by first-wavelength light, the reporter molecules will fluoresce at the second wavelength.

Further aspects provide a third apparatus for detecting the presence or absence of a nucleic acid within a reaction container. The third apparatus comprises a support structure shaped and dimensioned to hold the reaction container so that contents of the reaction container occupy a first region in space; a support structure shaped and dimensioned to hold a reference tube so that contents of the reference tube occupy a second region in space; an infrared light source aimed to impart equal amounts of infrared radiation on the reaction container and the reference tube; a thermometer positioned to measure the temperature of material within the reference tube and generate an output indicative of the measured temperature; a first-wavelength light source aimed in a first direction so as to illuminate at least a first portion of the first region in space; and a light detector aimed in a second direction, wherein the light detector detects second-wavelength light arriving from the first portion of the first region in space. The first and second wavelengths are selected such that when reporter molecules are illuminated by first-wavelength light, the reporter molecules will fluoresce at the second wavelength.

### BRIEF DESCRIPTION OF DRAWINGS

FIG. 1 shows an exemplary CAD design of an apparatus for operational use in performing nucleic acid detection tests (e.g., to detect COVID-19);

FIG. 2 shows an exemplary workflow for operating a nucleic acid detection test including placing a swab in a tube containing reaction mix, placing the tube in the exemplary instrument shown in FIG. 1, and uploading the results to a data cloud using an app on a handheld device;

FIG. 3 shows the results of nucleic acid amplification of spiked SARS-CoV-2 RNA using a traditional thermocycler using an exemplary fast thermocycling protocol from a combined RT-PCR mix in presence of gold nanoparticles;

FIG. 4 shows an example of light-controlled thermocycling (left) Schematic diagram of light-controlled thermocycling, which consists of photothermal heating driven by an infrared LED acting on gold nanoparticles, and a contactless thermometer that reads the temperature of the mixture and data showing 30 highly controlled temperature cycles can be achieved in this example;

FIG. 5 shows the exemplary results from precise control of thermocycling times, and amplification of SARS-CoV-2 nucleocapsid gene;

FIG. 6 provides a table showing two exemplary amplification method schemes for testing two prototype instruments;

FIG. 7 shows a schematic diagram of one embodiment of an optical circuit for heating the contents of a reaction container and detecting the presence of specific proteins within the reaction container;

FIG. 8 shows an example of a hardware implementation that corresponds to the FIG. 7 schematic diagram;

FIG. 9 depicts the geometry of certain components in the FIG. 8 example, with other components omitted to more clearly show the geometry of the depicted components;

FIG. 10 shows an exemplary schematic depicting the user steps involved in an ultrafast SARS-CoV-2 molecular test;

FIG. 11 shows an exemplary workflow overview for an ultrafast SARS-CoV-2 molecular test;

FIG. 12 shows the results of experiment testing the effect of different concentrations of gold nanoparticles (“Au-NRs”) on heating rates;

FIG. 13 shows the results of an experiment examining the effects of presence of Au-NPs on inhibition of the raw fluorescent signal (left) and accuracy of nucleic acid amplification;

FIG. 14 shows improved amplification from complementary DNA (“cDNA”) reversed transcribed from COVID RNA;

FIG. 15 shows reverse transcribed (RT) PCR (“RT-PCR”) (Left) on an exemplary nucleic acid detection system showed successful amplification (p<0.0001 compared to no template controls (“NTCs”) and normalized endpoint fluorescence values from individual P1 +RNA runs showing results for both FAM (N1 target) and HEX (SPC) (Right);

FIG. 16 shows RT-PCR with different starting copy numbers;

FIG. 17 shows the results of reducing the time of reverse transcriptase step showing minimal difference between 1-minute, 3-minute, and 5-minute steps;

FIG. 18 shows exemplary testing of 1-minute RT-PCR with successful amplification compared to NTCs;

FIG. 19 shows exemplary ultrafast cycling with 1-minute RT step and no holds during temperature cycling achieves <15 minute amplification with a limit of detection of 104 copies/mL from crude saliva lysates of SARS-CoV-2 clinical samples;

FIG. 20 provides the results of an experiment showing endpoint fluorescence (left) and Ct value (right) for RT-PCR from SARS-CoV-2 clinical samples;

FIG. 21 shows successful detection of positive SARS-CoV-2 clinical samples compared to no template controls (NTCs);

FIG. 22 shows the results of an exemplary assessment of three instruments for detection of positive SARS-CoV-2 clinical samples;

FIG. 23A depicts the steps of an exemplary process for implementing real-time, simultaneous detection of multiple targets in a single sample tube through heating, cooling, and measurement phases of a PCR cycle;

FIG. 23B depicts an example of a suitable set of components for implementing the steps depicted in FIG. 23A;

FIG. 23C depicts an example of a suitable set of components for implementing the multispectral fluorescence monitoring step depicted in FIG. 23A;

FIG. 23D is a graph depicting optical spectra of 3 molecules, IR heating, 488 nm excitation, and Au-NR absorbance;

FIG. 24A shows well-controlled temperature cycling over a full RT-PCR sequence in less than 15 minutes;

FIG. 24B shows a graph of consistent heating and cooling rates achieved through 45 RT-PCT cycles;

FIG. 24C shows positive amplification results using the illustrated RT-PCR system;

FIG. 24D shows exemplary photothermal amplification LoD data using purified RNA and fast amplification (<15 minutes);

FIG. 25 shows exemplary positive and negative results using human saliva SARS-CoV-2 to validate the exemplary extraction-free workflow with plasmonic thermocycling;

FIG. 26A shows that addition of Au-NPs (OD18) to RT-PCR reactions quenches the raw fluorescent signal;

FIG. 26B shows exemplary Ct values derived from the data in FIG. 26A;

FIG. 26C shows multi-spectral detection using a single excitation laser and spectrometer;

FIG. 26D shows endpoint fluorescence detection in the presence of Au-NPs;

FIG. 26E shows real-time amplification and detection through both raw spectral curves increasing over time;

FIG. 26F shows exemplary plots of deconvolved fluorescence values (for three individual targets/colors) against cycle number to calculate Ct values;

FIG. 27A shows real-time amplification curves were detected across a four-log range of concentrations;

FIG. 27B shows exemplary Ct values for human RP and SARS-CoV-2 N1 across each concentration tested;

FIG. 27C shows exemplary N1 Ct values from prototype runs versus;

FIG. 27D shows clinical specimen testing for 11 positive specimens and 10 negative samples;

FIG. 27E shows an exemplary receiver operating characteristic curve for clinical specimens tested in FIG. 27D; and

FIG. 27F shows an exemplary comparison of N1 Ct values from the illustrated apparatus herein versus N1 Ct values from the same samples run on laboratory-based PCR.

### DETAILED DESCRIPTION OF THE PREFERRED EMBODIMENTS

For widespread use, current point-of-care molecular tests are either too expensive and insensitive or too expensive, slow, and complex. There is an urgent need for an affordable, fast, and simple-to-use molecular tests, in a manner that avoids chaotic and socially dense queues at limited testing sites.

Methods and apparatus described herein perform highly sensitive and reliable nucleic acid detection (e.g., reverse transcriptase polymerase chain reaction or RT-PCR) in a fast, lower cost, and integrated way. In some instances, the apparatus and method requires only a single step from a user perspective, and the result can be generated using an app in less than ten minutes. In some instances, the exemplary apparatus and system uses photothermal amplification based on the rapid absorption of infrared light by gold nanoparticles (Au-NPs).

Aspects described herein are faster, more accurate, and less expensive that previous point-of-care molecular diagnostic tests. In addition, they avoid contamination from direct contact with thermocouples, and integrate reagents and sample preparation into a single reaction chamber. In some instances, the methods and apparatus can be used for rapid diagnosis of SARS-CoV-2, but the methods and apparatus can be used for any point-of-care molecular nucleic acid testing by using primer sets designed to detect other target molecules.

In one aspect, the method and apparatus utilizes 1) single-step sample preparation using a temperature-release mechanism for reagents rather than mechanical switches, 2) ultrafast light-controlled thermocycling (both photothermal control and contactless measurements with closed-loop software feedback control), and 3) an industry-level secure and scalable software platform (both in mobile app that controls the instrument, and connection to a cloud server).

Reagents used in the apparatus can be provided, for example, in a capsule having a temperature-sensitive seal (e.g., containing Mastermix and enzymes for PCR). In this aspect, reagents can be added directly to a swab collection medium without complicated mechanical components to eliminate the need for mechanical components to add sample processing reagents.

Fast thermocycling can be performed using an infrared thermometer for temperature feedback with real-time fluorescence readout of the amplified products. In some instances, a “temperature control” tube can be used to control thermal cycling. Instead of using a thermometer (contact or contactless) on the PCR tube itself, temperature measurements can be made using a “temperature control” tube that is positioned equidistant from the focal point of the IR LED+Lens as compared to the main PCR tube as described herein. The temperature control can be calibrated such that the temperature in the reference tube matches or coordinates to that of the PCR tube.

Aspects described herein provide a first method of detecting a nucleic acid in a single reaction chamber by (a) obtaining a patient specimen suspected of comprising a first nucleic acid, (b) forming a crude lysate from the patient specimen, (c) forming a reaction mixture by combining the crude lysate with infrared absorbing materials, a detecting nucleic acid, and at least one reporter molecule in the single reaction chamber, (d) heating the reaction mixture to at least 35° C. by irradiating the reaction mixture with infrared light, and (e) detecting a presence of the at least one reporter molecule, wherein a presence of the at least one reporter molecule indicates the patient specimen contains the first nucleic acid, and wherein steps (b) through (d) occur in the single reaction chamber. In some instances, the at least one reporter molecule comprises at least two reporter molecules.

In some instances, the infrared-absorbing material comprises gold nanoparticles. The term “infrared-absorbing material” refers to a gas, liquid, or solid that is capable of substantially absorbing infrared radiation It is understood that other suitable infrared-absorbing materials can be used including, but not limited to, metallic nanofilms.

The term “nanoparticles” refers to a particle of matter that is between about 1 and 500 nanometers in diameter.

The term “nucleic acid” includes deoxyribonucleic acid (“DNA”), ribonucleic acid (“RNA”), and variants of DNA and RNA including, but not limited to, miRNA, mRNA, cDNA, etc.

The term “crude lysate” refers to an unfiltered mixture of a patient specimen and reagents used in a process for detecting a nucleic acid where the reagents can disrupt or lyse cell membranes or tissue from a patient specimen or sample. No RNA extraction step is applied to a crude lysate for “cleaning up” the lysed mixture. For example, the virus is lysed (e.g. by temperature), and the DNA polymerase acts directly on the crude lysate mixture.

The term “reporter molecule” refers to a molecule that is detectable and measurable and can be used to monitor the presence of or level of expression of molecule of interest (e.g., nucleic acid, protein). As used herein, a reporter molecule can refer to a nucleic acid that is labelled with a detectable moiety (e.g., fluorescent moiety, radiolabel, etc.) to indicate the presence of a nucleic acid of interest. In some instances, more than one reporter molecule can be detected.

In some instances, the first nucleic acid is amplified using one of the polymerase chain reaction (PCR) or isothermal amplification. The term “PCR” refers to a method amplifying nucleic acid by a cyclical, multi-step process of heating double-stranded nucleic acid until the two strands separate, binding or annealing a complementary nucleic acid to a region of nucleic acid to be amplified, and extending the annealed nucleic acid strand to form two double-stranded nucleic acid molecules, and cooling the nucleic acid.

In some instances, the isothermal amplification comprises loop-mediated isothermal amplification (LAMP).

The term “isothermal amplification” refers to a nucleic acid amplification technique that can be carried out without the need for thermocycling and with enzymes to operate at one constant temperature. Loop-mediated isothermal amplification (LAMP) is one example of an isothermal amplification technique.

In some instances, the heating of the reaction mixture denatures the first nucleic acid at a denaturing temperature. The term “denature” refers to separation of two complementary nucleic acid strands.

In some instances, the reaction mixture is cooled to an annealing temperature after and the detecting nucleic acid can anneal to the first nucleic acid, forming an annealed nucleic acid. The term “annealing temperature” refers to a temperature at which two complementary nucleic acid strands can bind to each other.

In some instances, a temperature within the reaction chamber cycles between a denaturing temperature and an annealing temperature at least 10 times. The term “cycle” refers to a repetition of the temperature adjustment between the denaturing and annealing temperatures such that a target nucleic acid can be amplified in an amount sufficient to be detected.

In some instances, nucleotides are added to or part of the reaction mixture, and an annealed nucleic acid can be extended along a complementary strand with the nucleotides.

In some instances, the first nucleic acid is ribonucleic acid (RNA). In some instances, the RNA is derived from a virus (e.g., SARS-CoV-2).

In some instances, the RNA is reverse transcribed prior to the heating of the reaction mixture. The term “reverse transcribed” refers to synthesis of DNA from RNA to form cDNA (complementary DNA).

In some instances, the reverse transcribing of the RNA occurs in about 1 to 5 minutes. In some instances, the heating step occurs in about 1 to 30 seconds. In some instances, the cooling step occurs in about 2 to 30 seconds. In some instances, the annealing and extending of the detecting nucleic acid occurs in about 1 to 60 seconds. In some instances, the detecting of the presence of the reporter molecule occurs in about 1 to 30 seconds.

Some instances further comprise filtering the crude lysate in the single reaction chamber.

Aspects described herein provide a second method for detecting a presence or absence of a plurality of different molecules within a reaction container comprising (a) illuminating contents of the reaction container using infrared light until a temperature within the reaction container reaches a denaturing temperature; (b) allowing the heated contents of the reaction container to cool until a temperature within the reaction container reaches an annealing temperature; (c) illuminating the contents of the reaction container with excitation light; (d) obtaining, while the contents of the reaction container are being illuminated with the excitation light, a respective measured spectrum of light that is being emitted by the contents of the reaction container; (e) deconvolving the respective measured spectrum into a plurality of respective individual spectra, each of which corresponds to a respective one of the different molecules; and (f) outputting data corresponding to each of the respective individual spectra. Steps (a) through (f) are repeated at least 10 times.

In some instances of the second method, step (g) comprises repeating steps (a) through (f) at least 40 times.

In some instances of the second method, the plurality of different molecules comprises at least three different molecules. In some instances of the second method, the plurality of different molecules comprises FAM, SUN, and ROX. In some instances of the second method, the plurality of different molecules comprises at least two molecules selected from the group consisting of FAM, SUN, HEX, and ROX. In some instances of the second method, each of the plurality of different molecules comprises a fluorescent dye having an excitation wavelength between 480 and 600 nm and an emission wavelength between 500 and 625 nm. In some instances of the second method, the reaction container contains gold nanoparticles dispersed in a liquid.

In some instances of the second method, step (a) comprises illuminating contents of the reaction container using infrared light for a first fixed interval of time; and step (b) comprises allowing the heated contents of the reaction container to cool for a second fixed interval of time.

Aspects described herein provide a first apparatus for detecting a presence or absence of a plurality of different molecules within a reaction container (40). The first apparatus comprises an infrared light source (50) aimed to illuminate contents of the reaction container; an excitation light source (95) positioned to illuminate contents of the reaction container; and a spectrometer (75) positioned to detect emission light emanating from the reaction container during times when the excitation light source is illuminating the contents of the reaction container. The first apparatus also comprises a controller programmed to, for each of N cycles, (a) control the infrared light source so that the temperature within the reaction container cycles between a denaturing temperature and an annealing temperature, (b) obtain, from the spectrometer, a respective measured spectrum in response to the excitation light, (c) deconvolve the respective measured spectrum into a plurality of respective individual spectra, each of which corresponds to a respective one of the different molecules, and (d) output data corresponding to each of the respective individual spectra, wherein N is an integer greater than or equal to 10.

In some embodiments of the first apparatus, N is greater than or equal to 40. In some embodiments of the first apparatus, at least one optical fiber (130) is used to route emission light from the reaction container to the spectrometer. In some embodiments of the first apparatus, the plurality of different molecules comprises at least three different molecules. In some embodiments of the first apparatus, the plurality of different molecules comprises FAM, SUN, and ROX. In some embodiments of the first apparatus, the plurality of different molecules comprises at least two molecules selected from the group consisting of FAM, SUN, HEX, and ROX. In some embodiments of the first apparatus, each of the plurality of different molecules comprises a fluorescent dye having an excitation wavelength between 480 and 600 nm and an emission wavelength between 500 and 625 nm.

Some embodiments of the first apparatus further comprise the reaction container (40), wherein the reaction container contains gold nanoparticles dispersed in a liquid. Some embodiments of the first apparatus further comprise the reaction container (40), wherein the reaction container contains nanoparticles dispersed in a liquid, where the nanoparticles are efficient absorbers of infrared light.

In some embodiments of the first apparatus, the control of the infrared light source comprises open-loop control based on time.

Further aspects provide a second apparatus for detecting the presence or absence of a nucleic acid within a reaction container (40). The reaction container has a longitudinal axis. The second apparatus comprises a support structure (45) shaped and dimensioned to hold the reaction container (40) so that contents of the reaction container (40) occupy a region in space; an infrared light source (50) aimed in a first direction so as to illuminate at least a first portion of the region in space; an infrared thermometer (55) aimed in a second direction so as to obtain temperature readings from at least a second portion of the region in space; a first-wavelength light source (60) aimed in a third direction so as to illuminate at least a third portion of the region in space; and a light detector (70) aimed in a fourth direction, wherein the light detector detects second-wavelength light arriving from the third portion of the region in space. The first and second wavelengths are selected such that when reporter molecules are illuminated by first-wavelength light, the reporter molecules will fluoresce at the second wavelength.

Some embodiments of the second apparatus further comprise a controller programmed to control the infrared light source based on a signal from the infrared thermometer so that the temperature within the reaction container (40) cycles between a denaturing temperature and an annealing temperature at least 10 times.

In some embodiments of the second apparatus, the second direction is substantially perpendicular to the first direction. In some embodiments of the second apparatus, the third direction is substantially perpendicular to the fourth direction.

In some embodiments of the second apparatus, the longitudinal axis of the reaction container (40) and the first direction are offset by 40-50°. In some embodiments of the second apparatus, the longitudinal axis of the reaction container (40) and the first direction are offset by 25-70°.

In some embodiments of the second apparatus, the second direction is substantially perpendicular to the first direction, the third direction is substantially perpendicular to the fourth direction, and the longitudinal axis of the reaction container and the first direction are offset by 40-50°.

Some embodiments of the second apparatus further comprise a fan (80) aimed in a fifth direction to blow at the region in space. The fifth direction can be substantially perpendicular to the first direction. The fifth direction can also be substantially perpendicular to the second direction.

Some embodiments of the second apparatus further comprise the reaction container (40), wherein the reaction container (40) contains gold nanoparticles dispersed in a liquid. Some embodiments of the second apparatus further comprise the reaction container (40), wherein the reaction container (40) contains nanoparticles dispersed in a liquid, and where the nanoparticles are efficient absorbers of infrared light.

In some embodiments of the second apparatus, the light detector also detects third-wavelength light arriving from the third portion of the region in space, wherein the third wavelength is different from the second wavelength.

Further aspects provide a third apparatus for detecting the presence or absence of a nucleic acid within a reaction container. The third apparatus comprises a support structure shaped and dimensioned to hold the reaction container so that contents of the reaction container occupy a first region in space; a support structure shaped and dimensioned to hold a reference tube so that contents of the reference tube occupy a second region in space; an infrared light source aimed to impart equal amounts of infrared radiation on the reaction container and the reference tube; a thermometer positioned to measure the temperature of material within the reference tube and generate an output indicative of the measured temperature; a first-wavelength light source aimed in a first direction so as to illuminate at least a first portion of the first region in space; and a light detector aimed in a second direction , wherein the light detector detects second-wavelength light arriving from the first portion of the first region in space. The first and second wavelengths are selected such that when reporter molecules are illuminated by first-wavelength light, the reporter molecules will fluoresce at the second wavelength.

Some embodiments of the third apparatus further comprise a controller. The controller can be programmed to control the infrared light source based on the output generated by the thermometer so that the temperature within the reference tube cycles between a denaturing temperature and an annealing temperature at least 10 times.

Some embodiments of the third apparatus further comprise a fan aimed to blow equal amounts of air on the reaction container and the reference tube.

I some embodiments of the third apparatus, the light detector also detects third-wavelength light arriving from the first portion of the first region in space, wherein the third wavelength is different from the second wavelength.

### EXAMPLES

### Example 1—Instrument

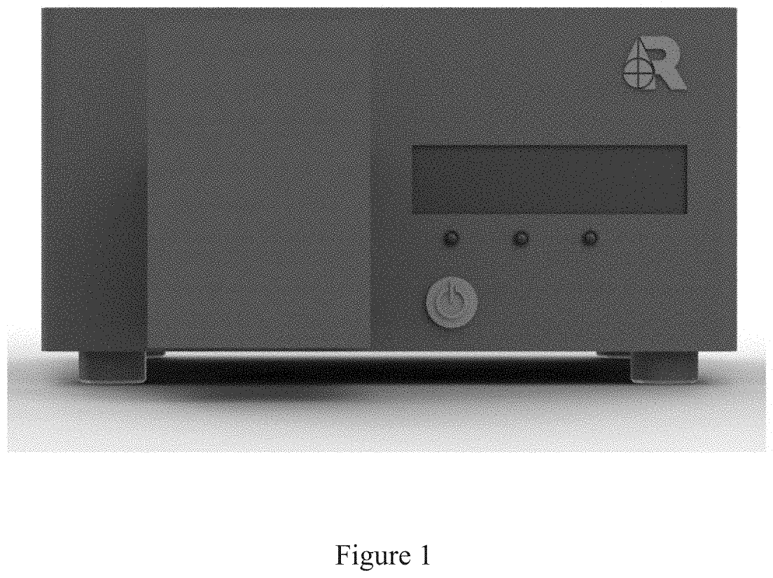

FIG. 1 shows an exemplary CAD design of an instrument with dimensions of about 4″×1.3″×4″. Disposable tests for COVID-19 or another target can be run on this instrument.

The exemplary instrument shown in FIG. 1 is designed to be affordable, fast, and simple to use. Because of the low cost of the instrument, it can be distributed across a large number of testing sites, such as companies, pharmacies, institutions, doctor's offices, and consumer's homes.

In the illustrated example, each instrument can run one test at a time. Optionally, multiple copies of the instrument may be used to enable multiple tests to be run in parallel within a benchtop space to achieve faster processing of multiple samples. Unlike high-throughput testing machines, tests are not processed in batches which lengthens the sample-to-result times, as testing of every specimen waits until the last specimen collected to initiate a single run. Instead, each test is processed in its own individual machine in real time, to maximize the fastest possible speed at which every individual can get their test result.

This high degree of scalability (across locations, speed, and throughput) also allows people to be tested frequently, facilitating, for example, same-day status changes to quarantine or back to work.

The aspects described herein also have the advantage of increased accuracy compared to other systems (as described below). For every percentage reduction in sensitivity, a COVID-19 patient that receives a false negative test not only poses a health risk to themselves and their neighbors, but also would result in testing re-visits for additional testing, hence placing a greater burden on national testing capacity.

Whereas RT-PCR has remained the gold standard for molecular testing since the 1980's, standard isothermal methods still lack the sensitivity, reliability and versatility among practical, commercially used nucleic-acid diagnostic techniques. The current point-of-care COVID-19 isothermal amplification devices face unresolved questions regarding low sensitivity.

More recently, CRISPR techniques are based on Cas enzymes, have been used. CRISPR techniques have demonstrated limited enzyme efficiency parameters at very low numbers of targets. Currently, CRISPR-based techniques are confined to a lab setting, and take over 40 minutes to generate a result. In addition, results using these systems have not yet been validated and proven for any diagnostic targets in a commercial, clinical, or a challenging point-of-care environment. In contrast, PCR methods have remained the gold standard for decades in terms of performance, and validated through use in numerous laboratories.

### Example 2—Connectivity

Optionally the exemplary apparatus described herein can be connected wirelessly (e.g. via Bluetooth) to a smart tablet or smartphone running an app that communicates with the instrument. Use of this or a similar connectivity system can enable a tight coupling between distributed, low-cost COVID-19 testing and cloud-based analysis of health data (including low-latency alerting of contacts, if desired) while maintaining data security and individual privacy, built to defense industry-security and performance standards.

### Example 3—Workflow

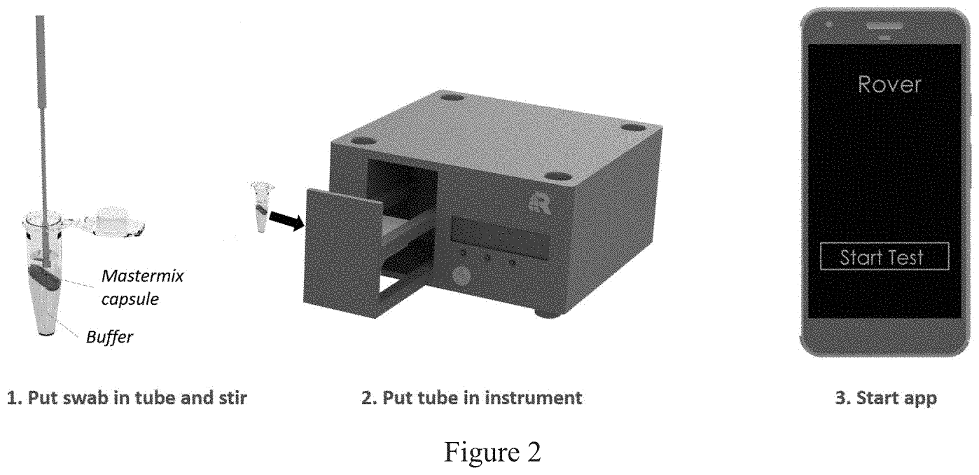

FIG. 2 shows an exemplary workflow for an exemplary COVID-19 test. In this example, the user places a nasal (or if necessary, nasopharyngeal) swab into a buffer that contains a capsule, stirs, and places the tube inside an instrument. Upon hitting “start” on an app, the instrument heats up the tube to the reverse transcription temperature, lysing the virus particles, and releasing, for example, the Mastermix (which contains enzymes, nanoparticles, primers, and concentrated reaction buffer) from the capsule. Reverse transcription and DNA amplification can takes place on the crude lysate. Alternatively, an additional membrane filtration step can be performed on the crude lysate within the reaction chamber for isothermal amplification.

After about 5 minutes, a high-positive result will be shown on the smart device; after about 12 minutes, a final result (positive or negative) is recorded on the app and sent to the cloud.

### Example 4—One-Step Sample Preparation

In the current COVID-19 pandemic, challenges exist with respect to low availability of disposable cartridges. In the FDA-authorized point-of-care RT-PCR systems, sample preparation is performed by complex cartridges, placing pressure on the supply chain and increasing the likelihood of quality assurance challenges.

The present apparatus and methods described herein utilize a sample preparation procedure that does not require complex cartridges. In this exemplary single-step, single reaction chamber approach for sample preparation (FIG. 2, step 1), a dry swab can be placed in 100 μL of Tris EDTA buffer (10 mM Tris-HCl at pH 7.5, 0.1 mM EDTA) that also contains gold nanoparticles and a small capsule (equivalent to less than 5 μL) containing, for example, Mastermix reagents inside a temperature-sensitive seal. A nasal swab can be used for high sensitivity testing. In another aspect, saliva can be used input specimen. Total sample volumes can be scaled down to less than 100 μL.

After stirring for 10 seconds, the tube is closed and placed into the instrument, and the user hits “start” on a mobile app (FIG. 2, steps 2 and 3). Initial ramp up for 2 to 4 minutes at the temperature of reverse transcriptase disrupts the seal and releases the Mastermix contents. The exemplary apparatus can be configured to automatically switch to the denaturation temperature to initiate fast PCR cycling. PCR has been previously shown to work on a crude SARS-CoV-2 lysate, for example by a group at Washington University ((doi: 2020.04.02.022384) Srivatsan et. al., Preliminary support for a “dry swab, extraction free” protocol for SARS-CoV-2 testing via RT-qPCR, bioRxiv. Preprint. 2020 Apr. 23 (https://www.ncbi.nlm.nih.gov/pmc/articles/PMC7263496/).

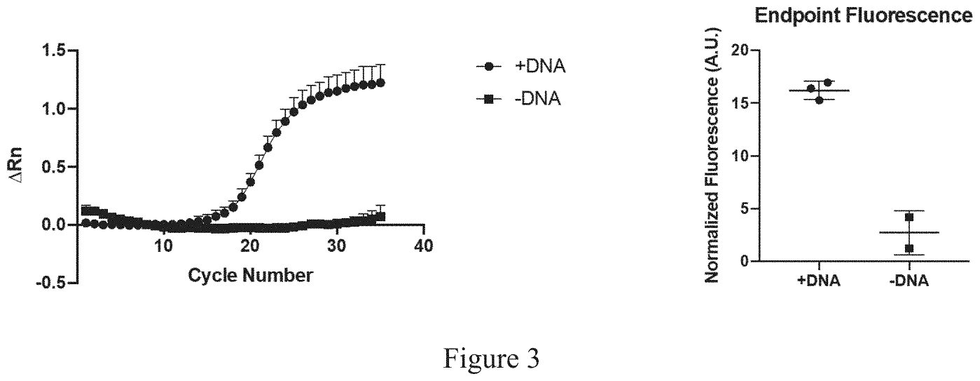

FIG. 3 shows the successful amplification (using traditional thermocycler) of spiked genomic SARS-CoV-2 RNA using a fast thermocycling protocol from a combined RT-PCR mix and in presence of gold nanoparticles. The left panel shows amplification of the target +DNA vs. −DNA in real time. The right panel show the endpoint fluorescence detected for the +DNA vs. −DNA. The data were collected on genomic SARS-CoV-2 RNA (BEI genomics) in the presence of nanoparticles and with a fast thermocycling protocol, on Quant Studio thermocycler.

SARS-CoV-2 genomic RNA (BEI resources) was successfully amplified with TaqPath One-Step RT-PCR master mix, which contains both the reverse transcriptase and DNA polymerase (FIG. 3). Gold nanoparticles were added to the reaction mixture, and the protocol was run in a traditional thermocycler with 5 minutes at 50 degrees C., 20 seconds at 95 C, and 45 cycles of 1 second at 95 degrees C. and 2 seconds at 55 degrees C. The endpoint fluorescence was measured using a plate reader. This exemplary experiment established the ability to perform a one-step RT-PCR on genomic RNA from SARS-CoV-2, in the presence of gold nanoparticles and with a slightly accelerated thermocycling protocol.

### Example 4—Fast, Light-Controlled Thermocycling

PCR instrumentation required to ramp up and down the temperature in a controlled manner can be limited by complexity, size, and cost. Standard thermal cycling, for example, uses a Peltier heating device, which requires a relatively large amount of power and time, and takes at least 40 minutes to complete the PCR cycles.

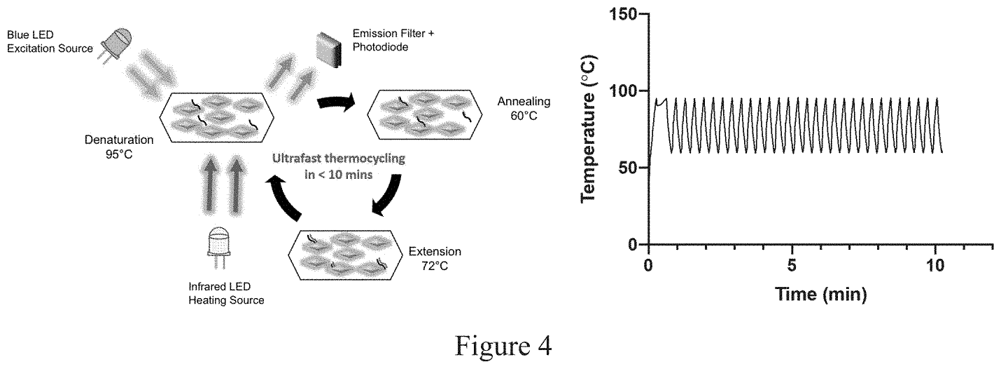

FIG. 4 shows an improved approach for implementing thermal cycling that relies on illuminating infrared-absorbing materials (e.g., gold nanoparticles) disposed within the reaction chamber with infrared light. The left panel provides a schematic diagram of light-controlled thermocycling comprising photothermal heating driven by an infrared LED acting on gold nanoparticles (or another alternative infrared-absorbing material), and a contactless thermometer that reads the temperature of the mixture. A feedback loop in the software adjusts the infrared LED in real time to achieve set temperatures. Standard fluorescence measurements can be taken simultaneously. The right panel shows that 30 highly controlled temperature cycles can be achieved in 10 minutes.

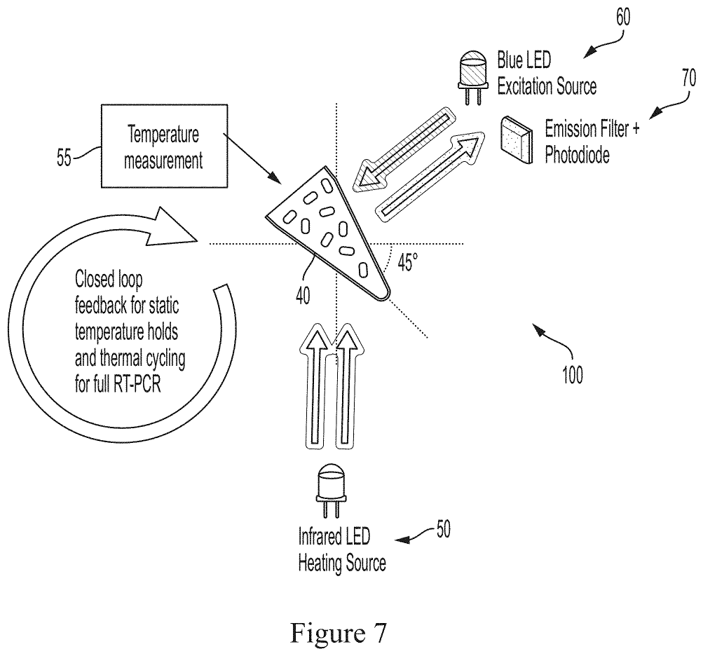

The apparatus and methods described herein replace the convective heating approach of a relatively large and expensive Peltier component with a photothermal process driven by compact optical components, and which drive thermocycling at 5 to 10 times faster than the conventional Peltier methods. In this exemplary setup (depicted in the left panel of FIG. 4, and described in further detail below in connection with FIGS. 7-9), gold nanoparticles in the mixture generate heat when exposed to IR light. When IR light is off, the nanoparticles stop generating heat, and optionally a compact fan cools the sample rapidly during temperature ramp down. A software performs the thermal cycling, measuring the temperature of the sample in the tube with an infrared thermometer. This setup can achieve ultrafast thermal cycling with 30 cycles in just 10 minutes (FIG. 4, right), or even in 5 minutes (e.g. heating and cooling cycles with 10 seconds of heating, and 4 seconds for cooling, data not shown).

### Example 5—Amplification

A modification of the RT-PCR test used the N1 primer and probe set to target the SARS-CoV-2 nucleocapsid gene. In this exemplary light-controlled setup for thermocycling, the one-step TaqPath RT-PCR kit (combined reverse transcriptase and DNA polymerase) was used in the reaction mixture with gold nanoparticles to achieve the thermocycling of the PCR step. Across triplicates, this optical setup achieved well-controlled thermocycling (FIG. 5, left).

In this experiment, short hold times were programmed (95° C. for 1 second, and 60° C. at 2 seconds), and achieved a precision of less than 3% in total cycling over 40 cycles. This exemplary RT-PCR setup successfully amplified and detected the SARS-CoV-2 nucleocapsid gene within a positive control plasmid (IDT), using the gold-standard CDC primer sequences (https://www.cdc.gov/coronavirus/2019-ncov/lab/rt-per-panel-primer-probes.html), at 2 copies/μL (FIG. 5, right).

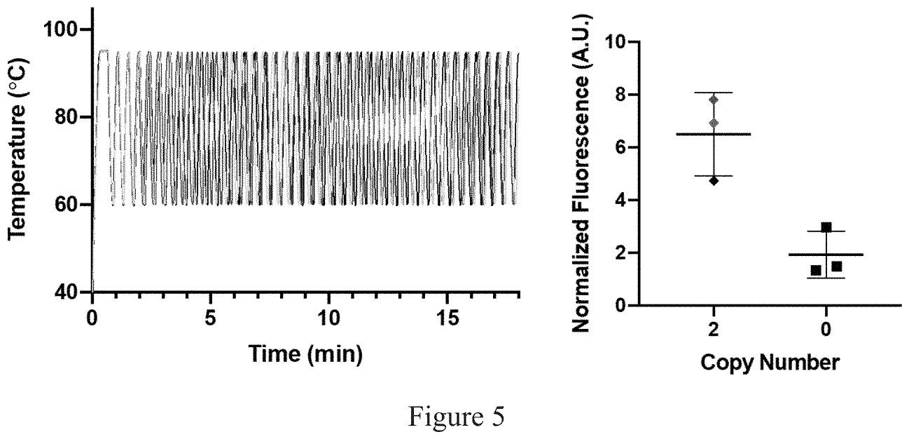

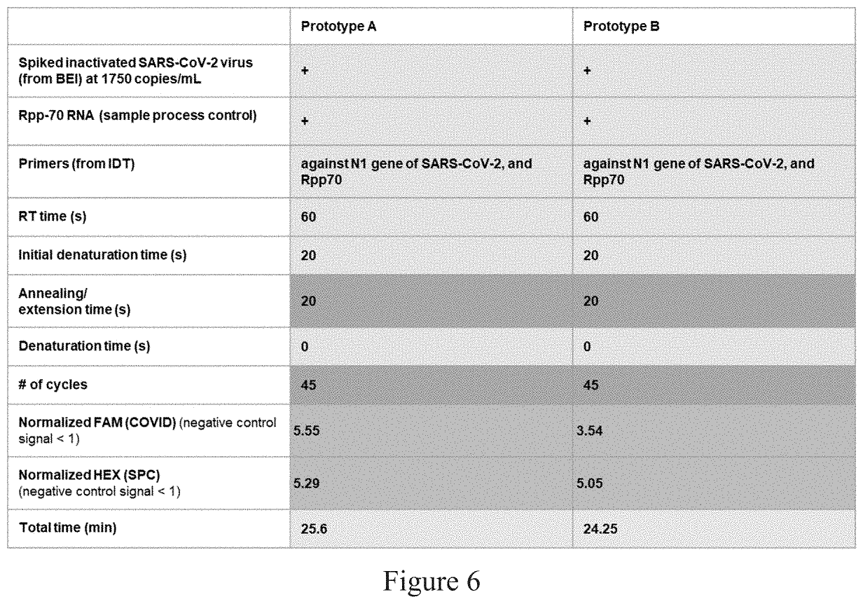

FIG. 6 describes the testing of two independent instruments. Human saliva was spiked with known concentrations of inactivated SARS-CoV-2 (obtained from BEI Resources). Data at 1750 copies/mL are shown here. Rpp-70 RNA which is already present in the human saliva was used as positive control. After 45 cycles of photothermal-induced thermocycling, fluorescence at two wavelengths were measured. Without RNA from SARS-CoV-2 or without the human RNA, the normalized signals were <1. Here, clear signals were detected for both the N1 gene of SARS-CoV-2 RNA and the Rpp-70 gene of human RNA.

### Example 6—Compact Instrument

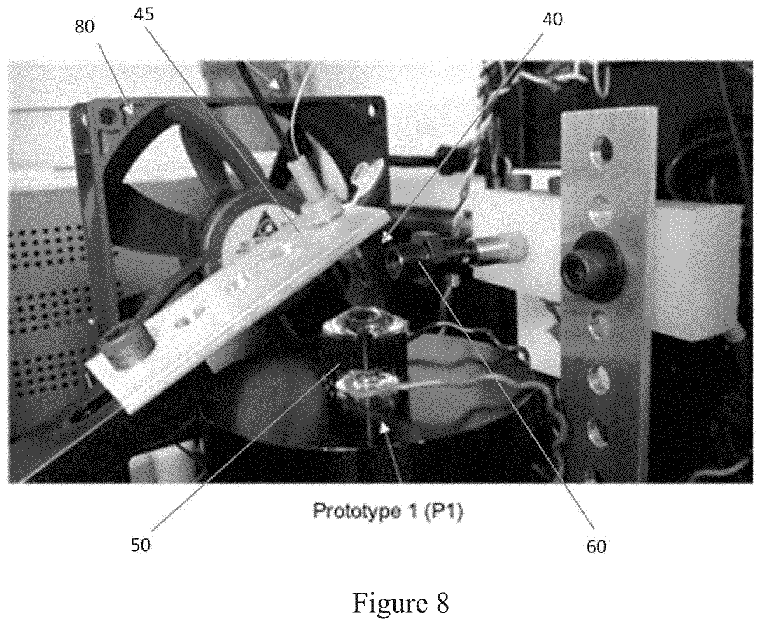

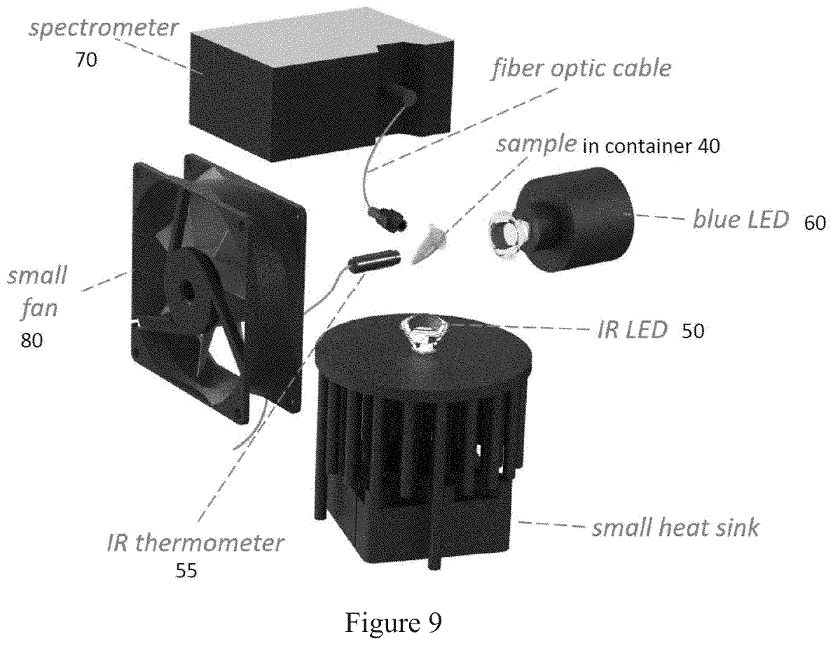

FIGS. 7-9 depict an embodiment of an apparatus 100 for heating the contents of a reaction container and monitoring its temperature (e.g., to perform thermal cycling) and detecting the presence of specific proteins within the reaction container by detecting a fluorescent signal generated by molecular probes. More specifically, FIG. 7 is an optical schematic diagram of this embodiment; FIG. 8 shows an example of a hardware implementation of this same embodiment; and FIG. 9 depicts the geometry of certain components in this same embodiment, with other components omitted to more clearly show the geometry of the depicted components.

The apparatus 100 detects the presence or absence of a nucleic acid within a reaction container 40, and this apparatus 100 may be used to implement the methods described herein. The reaction container 40 has a longitudinal axis. In some preferred embodiments, a thin-walled PCR tube is used as the reaction container 40. But in alternative embodiments, any of a variety of alternative reaction containers may be used. The apparatus 100 includes a support structure 45 that is shaped and dimensioned to hold the reaction container 40 so that contents of the reaction container 40 occupy a region in space.

The apparatus 100 also includes an infrared light source 50 aimed in a first direction so as to illuminate at least a first portion of the region in space (i.e., the interior of the reaction container 40). When the reaction container 40 contains an infrared absorbing material (e.g., gold nanoparticles) suspended in a fluid (e.g., PCR mastermix), illuminating the interior of the reaction container 40 with infrared light causes the contents of the reaction container 40 to heat up. In the FIGS. 7-9 embodiment, an 850 nm infrared (IR) LED serves as the infrared light source 50. The IR LED is attached to a heat sink and positioned vertically directly below the reaction container 40 and aimed straight up in a vertical direction. The longitudinal axis of the reaction container 40 is tilted at a 45° with respect to the vertical beam emanating from the IR LED to optimize fast heating rates. In alternative embodiments, the angle between the longitudinal axis of the reaction container and the beam emanating from the IR LED is between 40-50°, or even between 25-70°. Additionally, the beam emanating from the IR LED may deviate from the vertical.

The apparatus 100 also includes an infrared thermometer 55 aimed in a second direction so as to obtain temperature readings from at least a second portion of the region in space (i.e., the interior of the reaction container 40). Preferably, the second portion of the region in space coincides with the first portion of the region in space. But there may be some offset between those two regions. The infrared thermometer 55 may be aimed at the exact same spot within the interior of the reaction container 40 as the IR LED. Alternatively, the infrared thermometer could be aimed at a different spot within the interior of the reaction container 40. Any of a variety of approaches for implementing the infrared thermometer may be used, (e.g., Pyrometer Optris CS LT with CF lens and USB programming—https://www.optris.com/product-configurator-cs-lt). In the FIGS. 7-9 embodiment, the second direction is substantially perpendicular to the first direction. But in alternative embodiments, the angle between the second direction and the first direction may vary to some extent (e.g., within 30°)from perpendicular.

The apparatus 100 also includes a first-wavelength (excitation) light source 60 is aimed in a third direction so as to illuminate at least a third portion of the region in space. In the FIG. 7-9 embodiment, the first wavelength light source 60 is a blue LED with a peak output at 470 nm. The first-wavelength light source (e.g., the blue LED) serves as a fluorescence excitation source and it is aimed at the region in space (i.e., the interior of the reaction container 40). Note that the wavelength of the first-wavelength light source may vary from 470 nm, as long as it can excite fluorescence. In the FIG. 7-9 embodiment, the first-wavelength light source 60 is positioned so that the beam emanating from the light source 60 will be perpendicular to the longitudinal axis of the reaction container 40. But in alternative embodiments the angle between the longitudinal axis of the reaction container 40 and the beam emanating from the light source 60 will vary to some extent (e.g., within 30°) from perpendicular.

The apparatus 100 also includes a light detector 70 aimed in a fourth direction. This light detector 70 detects second-wavelength (emission) light arriving from the third portion of the region in space. The purpose of this light detector 70 is to detect fluorescent emmission light arriving from the portion of the reaction container 40 that is being illuminated by the excitation light from the first wavelength light source 60. In some embodiments, a photodiode may serve as the light detector 70. In some embodiments, the light detector 70 may be implemented using a collimating lens attached to a fiber optic cable that leads to a spectrophotometer. The first and second wavelengths are selected such that when reporter molecules are illuminated by first-wavelength light, the reporter molecules will fluoresce at the second wavelength. In the FIG. 7-9 embodiment, the third direction is substantially perpendicular to the fourth direction. But in alternative embodiments, the angle between the third and fourth directions (i.e., the angle between the beam emanating from the light source 60 and the aim of the light detector 70) will vary to some extent (e.g., within 30°) from perpendicular.

In some embodiments, the light detector 70 responds to more than one wavelength (e.g., by using a spectrometer as the light detector 70). For example, in addition to responding to second-wavelength light, the light detector 70 may respond to third-wavelength light (where the third wavelength is different from the second wavelength) and optionally additional wavelengths of light. These embodiments may be used to detect emission light at different wavelengths (e.g., second-wavelength light and third-wavelength light) from different reporter molecules. In these embodiments, deconvolution may be used to separate out the contribution of each of the detected wavelengths (e.g., as described below).

Optionally, a fan 80 (e.g., a 12 V rotary fan) is aimed to blow at the region in space (i.e. to blow at the reaction container 40) to speed up cooling of the contents of the reaction container 40. The fan 80 may be aimed in a fifth direction that is both (a) substantially perpendicular to the first direction and (b) substantially perpendicular to the second direction.

When a reaction container 40 is positioned at the region in space and the reaction container 40 is filled with a liquid in which gold nanoparticles (or another infrared-absorbing material) are dispersed, the contents of the reaction container 40 can be heated by activating the infrared light source 50, and the temperature within that reaction container 40 can be measured by monitoring signals received from the infrared thermometer 55.

A controller (not shown) controls the apparatus 100. In some embodiments, this controller is programmed to control the infrared light source 50 based on a signal from the infrared thermometer 55 so that the temperature within the reaction container 40 cycles between a denaturing temperature and an annealing temperature at least 10 times. The controller can implement a closed-loop feedback control system for IR-controlled heating and cooling by controlling the infrared light source 50 based on signals received from the infrared thermometer 55.

In some embodiments, temperature control and measurements can be run on LabView, and fed into a PID (proportional-integral-derivative) algorithm for IR heating and passive cooling or fan-assisted cooling. This control can be transferred e.g., via Bluetooth to a mobile app (e.g. Science Translational Medicine, doi: 10.1126/scitranslmed.aaa0056).

Current measurements indicate a limit of detection within an order of magnitude of the plate reader measurements shown previously in the amplification results. Optical alignment and electronics optimization can be performed to close this small gap.

In the FIGS. 7-9 embodiment described above, an infrared thermometer 55 is aimed to obtain temperature readings from the interior of the reaction container 40. But in alternative embodiments (not shown), an indirect approach is used to measure the temperature inside the reaction container 40 in place of the infrared thermometer. In these embodiments, the support structure is shaped and dimensioned to (a) hold the reaction container so that contents of the reaction container occupy a first region in space and (b) to hold a reference tube so that contents of the reference tube occupy a second region in space. The infrared light source is aimed to impart equal amounts of infrared radiation on the reaction container and the reference tube. Because of this, the temperature inside the reaction container can be estimated by measuring the temperature inside the reference tube (assuming that the reaction container and the reference tube have the same amount of liquid and the same amount of infrared-absorbing material). A thermometer is positioned to measure the temperature of material within the reference tube and generate an output indicative of the measured temperature. Any of a variety of conventional thermometers may be used to measure the temperature of the material within the reference tube including but not limited to a K-type wire gauge thermocouple placed inside the tube.

The controller determines the temperature inside the reaction container by using the output of the thermometer to measure the temperature in the reference tube, and then making an assumption that the temperature in the reaction container is the same as the temperature in the reference tube. When a fan is used for cooling in these embodiments, the fan should be aimed to blow equal amounts of air on both the reaction container and the reference tube.

A prototype apparatus can be constructed as follows. Infrared LED is positioned underneath a lens and set to directly shine onto the PCR tube. The LED/lens setup is attached to a heat sink and fan to prevent overheating.

The PCR tube was placed at a 45° angle and held in place using a breadboard and brackets (ThorLabs) and a metal adapter (machined in house). An additional fan was positioned perpendicular to the LED and used to cool the sample. K-type thermocouples (Omega) were used to measure temperature.

A 3D-printed ‘thermocouple guide’ was used to position the thermocouple in the PCR tube. A program written in LabView was used to measure temperature, and provide a feedback loop for thermocycling. The program used a PID algorithm to improve temperature holds. For amplification experiments, a thermocycling protocol of 20 seconds at 95° C. followed by 35-40 cycles of 95° C. for 1 second and 60° C. for 2 seconds was used.

Functionalized silica-coated gold nanorods (Au-NRs) were purchased from Nanopartz. Au-NRs had an SPR peak ˜855 (slight variation between batches) and an aspect ratio ˜4.5 nm. The silica coating was 10-nm thick. Amplification with Au-NRs in the mix was tested to demonstrate no inhibition of PCR. For initial compatibility testing, master mix consisting of 5 μL of TaqPath ProAmp MasterMix (ThermoFisher), 0.25 μL Taqman primer/probe mix (40× working concentration, ThermoFisher), 1.75 μL BSA (10 mg/mL in 1× PBS with 0.1% Tween-20), 0.5 μL template DNA (2.5 ng/μL working concentration, IDT) and Au-NRs (final OD 18) and nuclease-free water to a final volume of 10 μL. Standard thermocycling was performed per manufacturer's recommendation in a QuantStudio 3. Fast thermocycling was performed with a 3-minute initial denaturation at 95° C. followed by 40 cycles of 10 seconds at 95° C. and 15 seconds at 60° C.

For experiments involving plasmids, 2019-nCoV N Positive Control plasmid, Hs_RPP30 Positive Control plasmid, and N1 and RP primers and probes were purchased (IDT). The N1 primer/probe set was purchased as part of the 2019-nCoV CDC RUO kit, and the RP primers/probes were purchased individually with the HEX fluorophore instead of the FAM fluorophore (Table 1). RP primers/probes were resuspended in IDTE pH8.0 (IDT) buffer to a working concentration of 6.7 μM for the probe and 1.7 μM for each primer, as indicated in the CDC protocol. For PCR mastermix using plasmids, no BSA was used and 0.375 μL/reaction of primer probe mix was used. Control amplifications were conducted using a ‘fast’ thermocycling protocol which included 20 seconds at 95° C. followed by 35-40 cycles of 95° C. for 1 second and 60° C. for 2 seconds. Negative controls were tested using the same mix and conditions but without template.

TABLE 1
Primer and probe sequences for SARS-CoV-2
testing (from DC)

| Name | Sequence |
|---|---|
| 2019-nCoV_N1-F | GAC CCC AAA ATC AGC GAA |
| | AT (SEQ ID NO: 1) |
| 2019-nCoV_N1-R | TCT GGT TAC TGC CAG TTG |
| | AAT CTG (SEQ ID NO: 2) |
| 2019-nCoV_N1-P | FAM-ACC CCG CAT/ZEN/TAC |
| | GTT TGG ACC-3IABkFQ |
| | (SEQ ID NO: 3) |
| RP-F | AGA TTT GGA CCT GCG AGC |
| | G (SEQ ID NO: 4) |
| RP-R | GAG CGG CTG TCT CCA CAA |
| | GT (SEQ ID NO: 5) |
| RP-P | HEX-TTC TGA CCT/ZEN/GAA |
| | GGC TCT GCG CG-3IABkFQ |
| | (SEQ ID NO: 6) |

TABLE 2
Additional Sequences of Primers and Probes
for Detection of SARS-CoV-2 RNA.

| | |
|---|---|
| N1_forward primer | 5′-GAC CCC AAA ATC AGC GAA AT-3′ |
| N1_reverse primer | 5′-TCT GGT TAC TGC CAG TTG AAT |
| | CTG-3′ |
| N1_probe | 5′-FAM-ACC CCG CAT TAC GTT TGG |
| | TGG ACC-BHQ1-3′ |
| N2_forward primer | 5′-TTA CAA ACA TTG GCC GCA AA-3′ |
| N2_reverse primer | 5′-GCG CGA CAT TCC GAA GAA-3′ |
| N2_probe | 5′-SUN-ACA ATT TGC CCC CAG CGC |
| | TTC AG-BHQ1-3′ |
| RP_forward primer | 5′-AGA TTT GGA CCT GCG AGC G-3′ |
| RP_reverse primer | 5′-GAG CGG CTG TCT CCA CAA GT-3′ |
| RP_probe | 5′-ROX-TTC TGA CCT GAA GGC TCT |
| | GCG CG-BHQ-1-3′ |

Table 2 discloses SEQ ID NOS 1, 2, 7-10, 4, 5, and 11, respectively, in order of appearance.

For experiments involving RT-PCR, genomic RNA from SARS-CoV-2 was purchased (Isolate USA-WA1/2020, BEI Resources). Total control RNA (ThermoFisher) was used as an internal positive control. For reverse transcription and for RT-PCR, a master mix consisting of 2.5 μL TaqPath One-Step RT MasterMix, GC (ThermoFisher), 0.6 primer/probe mix, 1 μL RNA, Au-NRs diluted to a final concentration of OD 18, and RNase-free water to bring the reaction volume up to 10 μL was used. For just reverse transcription, a 15-minute reverse transcription step was performed at 50° C. in the QuantStudio 3. For full RT-PCR, the standard protocol was performed as described in the manufacturer's protocol, as well as a shorter protocol which involved either a 5-, 3-, or 1-minute reverse transcription step followed by 20 seconds at 95° C. and 40 cycles of 1 second at 95° C. and 2 seconds at 60° C. Negative controls were tested with the same mix and conditions but without SARS-CoV-2 RNA and either with and without total control RNA.

For experiments in the prototype (named P1), 10 μL of mix was used with 15-75 μL ChillOut Liquid Wax (Bio-Rad) to prevent evaporation. To test samples for fluorescence, wax was removed and disposed of and the samples were loaded into the plate reader (BioTek) and measured. All samples were normalized to ROX for analysis. For initial experiments on P1, the following thermal cycling protocol was used as controlled in LabView: 5 minutes at 50° C., 20 seconds at 95° C., and 40 cycles of 1 second at 95° C. and 2 seconds at 60° C. For 1-minute RT, the same protocol was used but 1 minute at 50° C. instead of 5 minutes. For ultrafast testing, 1 minute at 50° C. was programmed, followed by 20 seconds at 95° C., followed by a second LabView program which included 40 cycles of 0 seconds at 95° C. and 0 seconds at 60° C. with no PID algorithm involved during the cycles.

### Example 9—Clinical Specimen Testing of SARS-CoV-2 From Crude Saliva Lysates

De-identified clinical specimens were obtained under a protocol approved by the Columbia University Medical Center IRB (AAAT0100). Saliva specimens were obtained from Mirimus Foundation/SUNY Downstate and stored in −80° C. upon receipt. Mirimus had previously inactivated samples by incubating them at 95° C. for 5 minutes. For initial testing in QuantStudio, some samples were again heated for 5 minutes at 95° C. upon receipt. Samples were diluted 1:2 in 1× TE buffer (10 mM Tris-HCl, 1 mM EDTA) and a master mix containing 5 μL of TaqPath One-Step RT-PCR MasterMix, GC (ThermoFisher), 1 μL N1 primer/probe mix, 1 μL RP primer/probe mix, 3.33 μL Au-NRs (final OD 18), and 9.7 diluted sample was used.

A ‘fast’ RT-PCR step with a 5-minute reverse transcription step was performed as previously described in a QuantStudio 3. For testing of samples using ultrafast thermocycling on P1, samples were maintained at 4° C. upon receipt. Samples were diluted 1:1 in 1× TE buffer and heated for 30 minutes at 95° C. Mastermix was made using 2.5 μL of TaqMan Fast Virus 1-Step Master Mix, 3.1 μL Au-NRs (final OD 18), 0.6 μL N1 primer/probe mix, 0.6 μL RP primer/probe mix, and 3.2 μL diluted and heated sample (or nuclease-free water for NTCs). The ultrafast protocol (i.e. 1 minute at 50° C., 20 seconds at 95° C., and 40 cycles of 0 seconds at 95° C. and 0 seconds at 60° C.) was run and endpoint fluorescence was measured on the plate reader as previously described.

All statistics, including one-way ANOVA followed by Sidak's multiple comparison tests and Student's t-tests were performed in GraphPad Prism 8.

### Example 10—Ultrafast SARS-CoV-2 Molecular Test

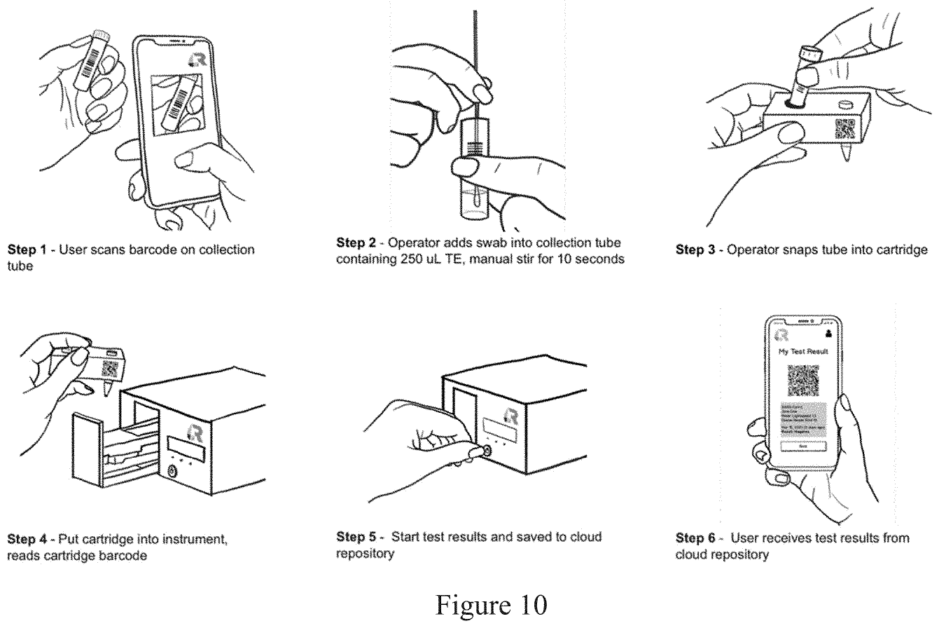

FIG. 10 depicts exemplary user steps for using the apparatus and methods described herein in a clinical setting. The apparatus and method can be used for walk-in testing in a pharmacy, or quick testing before entry into an office building, and thus the design facilitates simple, fast, and user-friendly steps. FIG. 10 depicts the user inserting a swab; however, the device was also designed to be compatible with saliva.

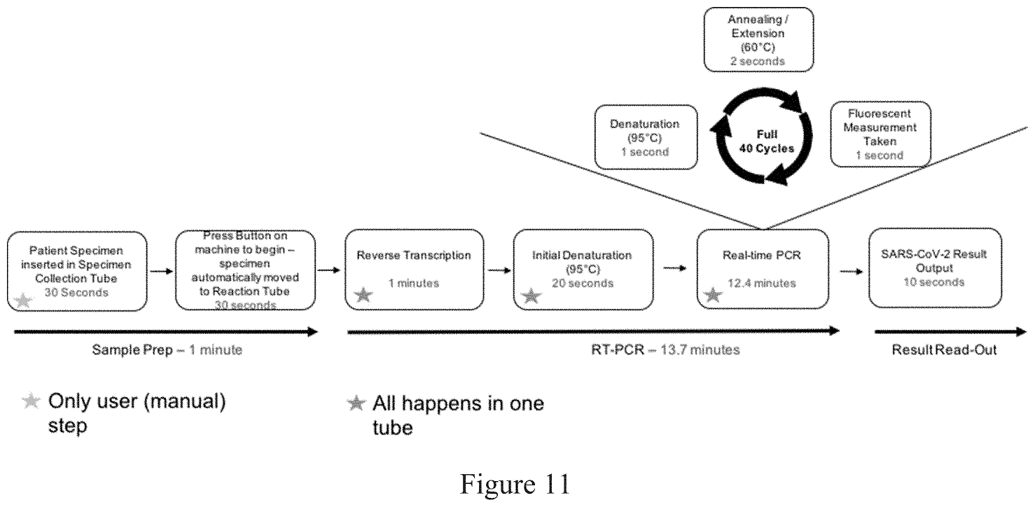

FIG. 11 describes the general workflow for the POC test. As shown in FIG. 11, a patient specimen is self-collected and inserted into the device. Once the user presses a button to start the run, the rest of the steps are automatically initiated within the device. This includes mixing the sample with a buffer and performing a full 40-cycles of RT-PCR using photothermal amplification. The device also is being designed to be compatible with real time fluorescent measurements, though the initial prototype uses an endpoint measurement. After approximately 15 minutes, a test result is sent to the user via a secure mobile platform.

### Example 12—Optimization of Nanoparticle Concentration

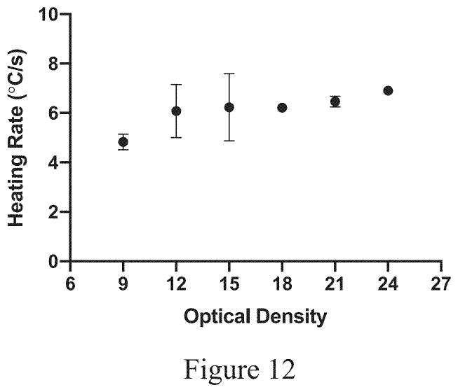

Initial testing with various concentrations of nanoparticles showed the ability to rapidly thermocycle in buffer (FIG. 12). An OD (optical density) of 18 for the nanoparticles was found to optimally enhance cycling times without significant variation and was the optimal concentration for a low-volume PCR test. FIG. 12 shows the effects of using different concentrations of Au-NRs on heating rates (error bars show S.D., n=5).

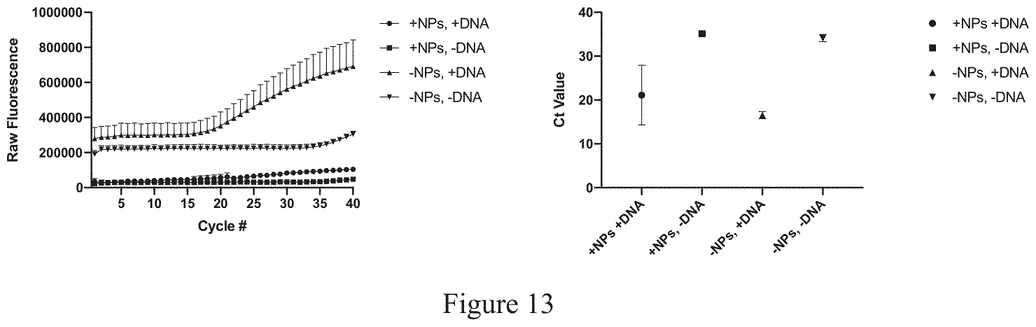

The effects of using of gold nanoparticles (Au-NPs) on inhibiting amplification during PCR is shown in FIG. 13. Although the Au-NPs quenched the raw fluorescent signal, the Au-NPs did not inhibit amplification and there was no significant difference between we were very clearly able to distinguish between reactions with DNA and the no template control (FIG. 13). While Au-NPs inhibit raw fluorescent signal (left), they do not inhibit the ability to accurately amplify nucleic acids (right).

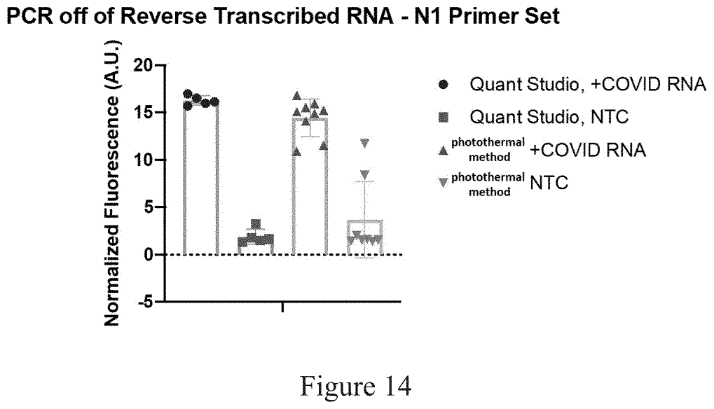

To determine which runs failed due to instrument failure, a ‘sample processing control’ (SPC) was added to each run. The addition of the SPC showed that there were priming issues with the plasmid. Thus, amplification was tested using reverse transcribed cDNA from human genomic SARS-CoV-2 RNA, which significantly improved the results compared to using plasmids (FIG. 14). Using this RNA, a 100% detection rate (starting copy number 5.5E5 copies/mL) was achieved.

### Example 13—Amplification From cDNA With Improved Results

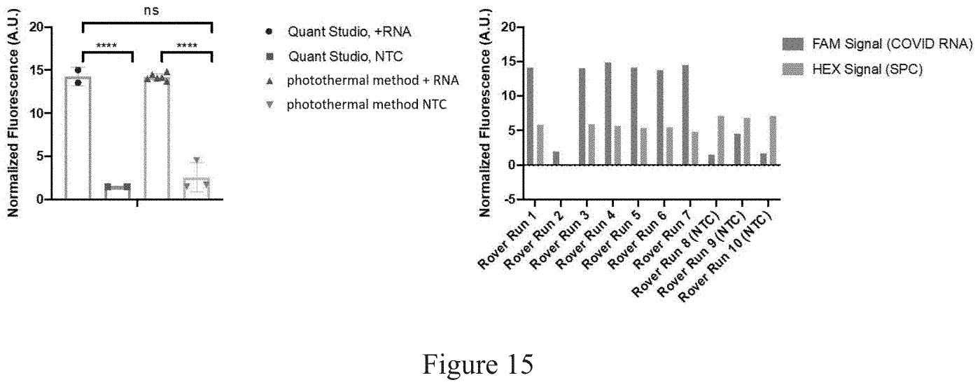

Full RT-PCR was performed using the exemplary P1 apparatus. Initial testing with 5.5E5 copies/mL showed a 100% accuracy rate (FIG. 15, left). Next, varying the starting copy numbers of purified RNA for RT-PCR in P1 was tested. FIG. 15 (Left) RT-PCR on the P1 system showed highly successful amplification (p<0.0001 compared to NTCs). Error bars show standard deviation. (Right) Normalized endpoint fluorescence values from individual P1+RNA runs showing results for both FAM (N1 target) and HEX (SPC). One SPC failure (Run 2) was detected out of 10 tests.

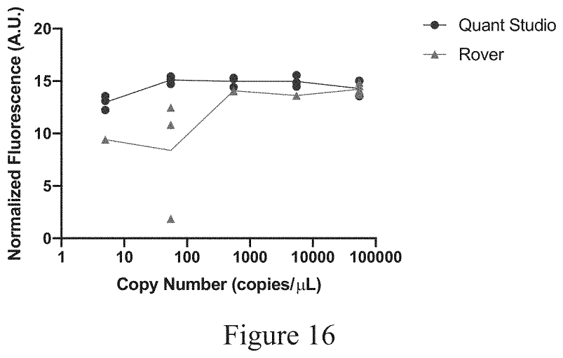

The apparatus was able to successfully detect down to 500 copies/mL (FIG. 16). FIG. 16 shows the results of RT-PCR with different starting copy numbers on the exemplary P1 apparatus. Initial testing showed detection down to 5.5 copies/μL in a 10 reaction (500 copies/mL).

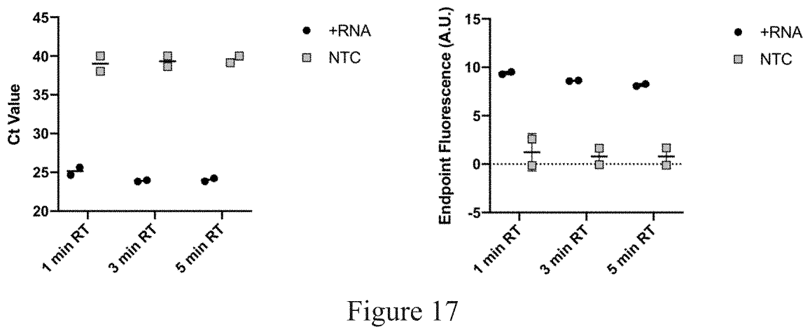

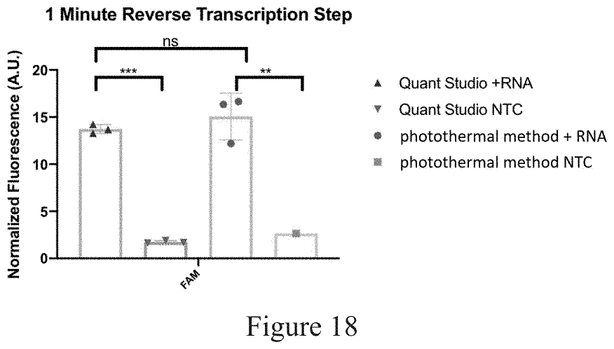

To further minimize the time needed for amplification, the ability to perform significantly faster reverse transcription than traditional protocols was tested. Comparing 15-minute, 5-minute, 3-minute, and 1-minute reverse transcription, there were no significant difference in Ct value or endpoint amplification (FIG. 17). One minute RT-PCR was performed on the exemplary P1 device and the results distinguished between positive and negative samples (FIG. 18).

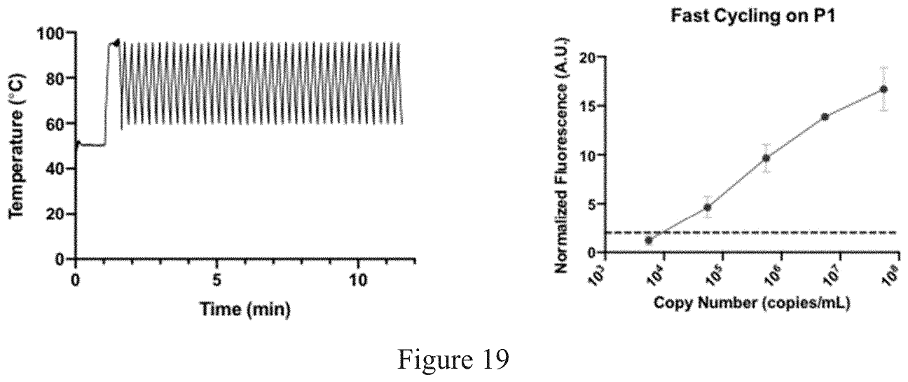

To even further reduce the time necessary for thermal cycling, the PID algorithm was removed during cycling (maintained during static temperature holds) and the holds at the top and bottom during cycling were removed (see materials and methods for exact times). The 1-minute RT step was also implemented. With these changes, we were able to achieve consistent positive results in an average time of 13.7±2.0 minutes (n=10). Here, the limit of detection was found to be 104 copies/mL (FIG. 19).

### Example 14—Clinical Specimen Testing of SARS-CoV-2 from Crude Saliva Lysates

Saliva samples from validated COVID-19 patients were tested using the exemplary apparatus and method using RT-PCR from crude lysates. Without being bound by theory, it is believed that due to the structure of SARS coronavirus capsids, which are self-assembled particles in which the lipid bilayer is a weak spot, that the virus could be lysed without the need for full RNA extraction. Because of the current shortage of RNA extraction kits, simple methods that avoid purification are advantageous (Vasudevan et al., Digital droplet PCR accurately quantifies SARS-CoV-2 viral load from crude lysate without nucleic acid purification, MedRxiv (https://doi.org/10.1101/2020.09.02.20186023); Lalli et al., Rapid and extraction-free detection of SARS-CoV-2 from saliva with colorimetric LAMP, Version 2. medRxiv. Preprint. 2020 May 11 [revised 2020 Aug 6](doi: 10.1101/2020.05.07.20093542); Lübke et al., Extraction-free SARS-CoV-2 detection by rapid RT-qPCR universal for all primary respiratory materials, Journal of Clinical Virology, Volume 130, September 2020 (https://doi. org/10.1016/j cv.2020.104579).

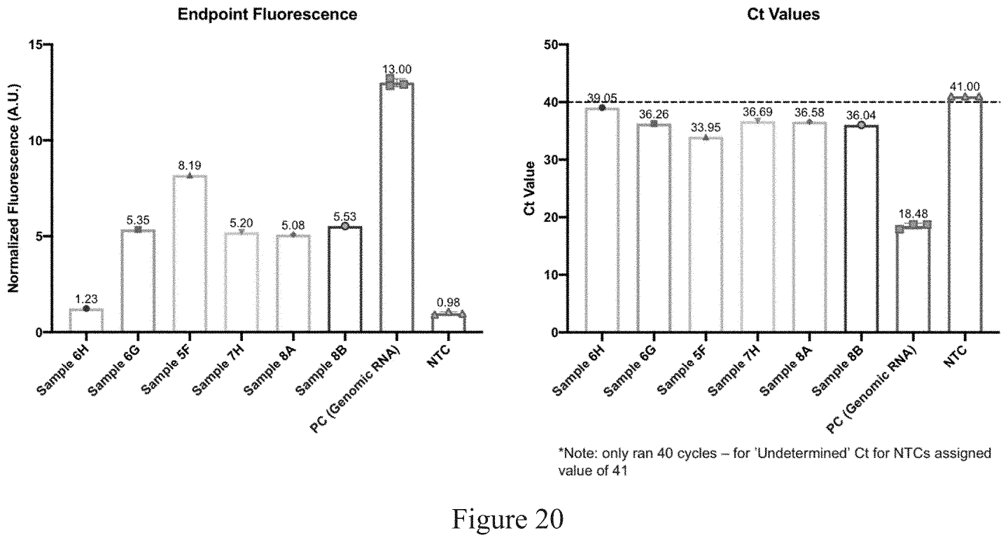

In one aspect, an additional 95° C. heat step was performed to assist in lysis in combination with dilution in TE buffer. Using the Ct (cycle threshold) value, 6/6 positive results were detected (FIG. 20, right panel). Using endpoint fluorescence, 5/6 positive results were detected (FIG. 20, left panel). Results showing endpoint fluorescence (left) and Ct value (right) for RT-PCR off of saliva specimens. 5/6 samples were identified as positive based on endpoint fluorescence and 6/6 samples were identified as positive based on Ct value.

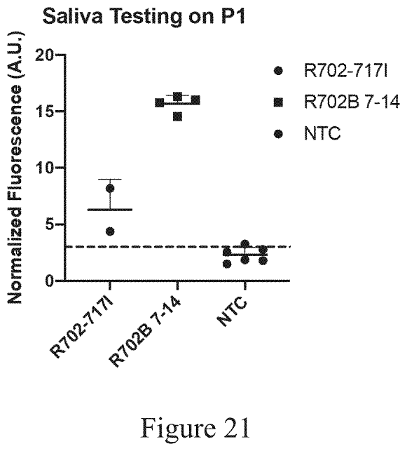

The ability to detect SARS-CoV-2 RNA in clinical saliva samples using the ultrafast protocol on the exemplary P1 apparatus was tested. Two saliva samples with known positive cases were prepared and tested on P1. For sample R702-7171, two replicates were conducted, and for sample R702B 7-14, four replicates were conducted. All samples tested positive via endpoint fluorescence with a cutoff threshold of 3.5 (FIG. 21). Tests took an average of 12.97±1.74 minutes and confirmed the known SARS-CoV-2 positive status of the clinical saliva samples. Initial clinical specimen testing on P1. Both samples were known positives and both samples tested positively for all replicates. Samples are compared to no template controls (NTCs).

The exemplary apparatus and methods disclosed herein are capable of ultrafast thermocycling and successful RT-PCR using photothermal amplification down to 500 copies/mL for slightly longer runs (<25 minutes) and down to 104 copies/mL for ultrafast runs (<15 minutes). These results also show the ability to perform RT-PCR from crude lysates from saliva samples and detection of SARS-CoV-2 RNA in saliva samples from clinical patients. In one example, the thermometer is a contactless IR thermometer.

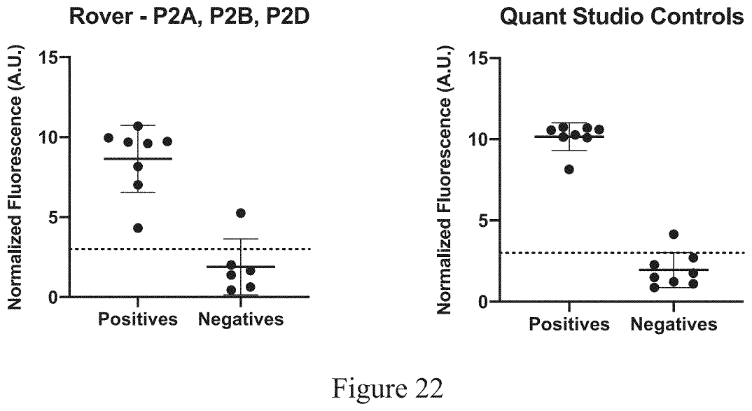

Eight positive saliva specimens were analyzed in duplicate and six negative saliva specimens were analyzed on three different instruments, as shown in FIG. 22. In this experiment, the instruments included two LEDs and 1 infrared thermometer to drive the thermocycling. A 10 uL final reaction volume (including unprocessed saliva specimen, Mastermix, primers, nanoparticles) was used. Eight positive saliva specimens were tested in duplicate, and 6 negative saliva specimens tested once. The results were compared to the results from an academic collaborator (shown on horizontal axis). The results from the exemplary device described herein are shown on the left panel and achieved 100% sensitivity (8/8) and 83% specificity (5/6). However, when compared to a QuantStudio instrument (right panel), the exemplary device described herein achieved 100% sensitivity and 100% specificity.

### Example 15—Infrared-Driven Plasmonic Thermocycling with Real-Time Multispectral Fluorescence Monitoring Using Small Optical Components

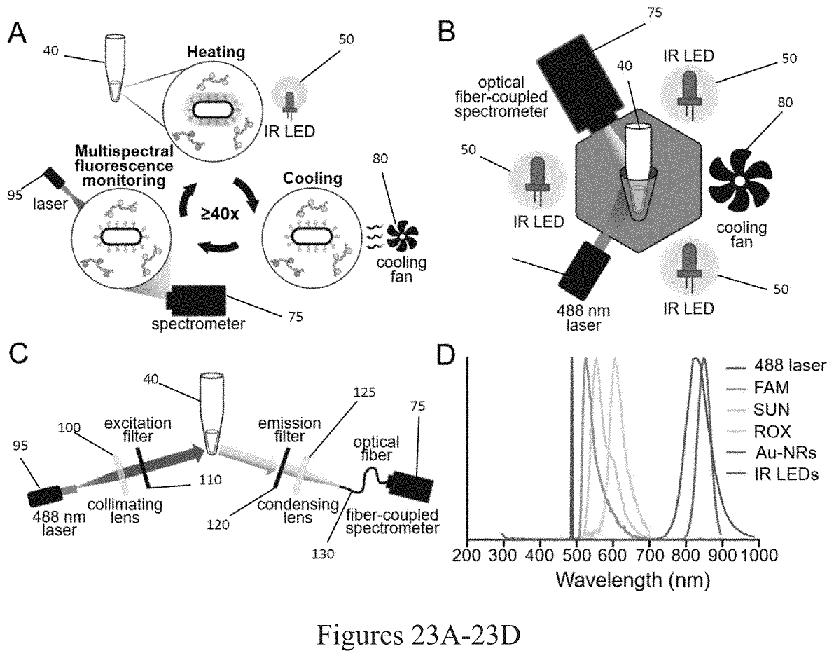

FIGS. 23A-D depict an overall concept and design of a multiplexed, real-time plasmonic RT-PCR for achieving both thermocycling at infrared wavelengths and multispectral fluorescence measurements at visible wavelengths. More specifically, FIG. 23A is a schematic diagram of multiplexed real-time plasmonic RT-PCR, with heating driven by infrared LEDs 50 acting on Au-NRs (i.e., gold nanorods) and cooling aided by a fan 80. The Au-NRs are suspended in solution in a 0.2 mL PCR tube 40, rapidly absorbing light from the LEDs 50 and converting it to heat, allowing for fast PCR thermal cycling. While gold nanorods are preferred, alternative nanoparticles may be used as long as they are able to function as absorbers of infrared light. A 488 nm laser 95 and spectrometer 75 setup are used to perform real-time fluorescent detection by taking a measurement at the end of each cooling cycle.

FIG. 23B depicts a CAD design of a suitable setup. A PCR tube 40 is surrounded by low-cost optical components, without Peltier heating elements. The main components of the instrument include a thin-walled PCR tube 40 surrounded by three IR LED modules 50, a cooling fan 80, and a 488 nm laser 95 and spectrophotometer 75 for fluorescent detection. The three IR LED modules 50 use 850 nm IR LEDs attached to heat sinks+heat sink fans and placed concentrically surrounding the PCR tube 40. Temperature control can be achieved through contactless closed-loop sensing with the IR thermocouple, closed-loop sensing with a wire thermocouple (not shown), or through open-loop control. This setup produced rapid heating throughout the tube compatible with RT-PCR.

FIG. 23C is a more detailed schematic of the fluorometer system. Light coming from a 488 nm laser 95 passes through a collimating lens 100 and an excitation filter 110 before reaching the PCR tube 40. Light emitted from the tube 40 passes through 500 nm edge emission filter 120 (e.g., Semrock) and a condensing lens 125 before traveling through an optical fiber 130 to reach the spectrometer 75.

FIG. 23D is a graph depicting non-overlapping optical spectra of various components within the system: 488 nm excitation peak, 3 emissions at 520 nm, 555 nm, and 610 nm, IR LED excitation and Au-NR absorbance.

In the FIG. 23A-D embodiment, gold nanorods (AuNRs) with a localized surface plasmon resonance in the near-infrared range (−850 nm) were used in the reaction vessels 40. This wavelength range allowed use of fluorescent probes for real-time fluorescence detection without the need to remove Au-NRs. A concentration of AuNPs that was sufficiently high (e.g., final OD 18) to achieve the photothermal effect without interfering with fluorescence measurements while rapidly generating heat throughout the entire solution (20 μL in volume) was used. During each cycle, cooling was achieved with a small 12V fan 80. The intensity and time sequence of the LEDs was calibrated using a K-type thermocouple and custom LabView program, which was fed into a second LabView program to automate tuning of an open-loop control system.

For real-time fluorescent monitoring, three common fluorescent probes (FAM, SUN, and ROX) were excited with a common excitation source and detection source (FIGS. 23C and 23D). In particular, with plasmon resonance of Au-NRs at near-infrared wavelength, a fluorescence setup using a 488 nm laser diode 95 as an excitation source was used along with an optical fiber-coupled spectrometer 75 for detection multiple wavelengths, and therefore multiple targets (FIG. 23D). In alternative embodiments, fluorescent dyes other than FAM, SUN, and ROX may be used (e.g., HEX, or another fluorescent dye having an excitation wavelength between 480 and 600 nm and an emission wavelength between 500 and 625 nm).

This setup uses small components, with a total cost of goods less than $1000 at scale to achieve real-time multispectral fluorescence monitoring alongside plasmonic thermocycling.

The FIG. 23A-D embodiment can be used to detect a presence or absence of a plurality of different molecules (e.g., dyes) within the reaction container 40. It uses an infrared light source 50 (e.g., one or more infrared LEDs) aimed to illuminate the contents of the reaction container. An excitation light source 95 (e.g., a 488 nm laser diode) is positioned to illuminate contents of the reaction container 40. A spectrometer 75 is positioned to detect emission light emanating from the reaction container during times when the excitation light source is illuminating the contents of the reaction container 40. For each of N cycles (e.g., at least 10 cycles or at least 40 cycles), a controller (not shown) controls the depicted components to (a) control the infrared light source 50 so that the temperature within the reaction container 40 cycles between a denaturing temperature and an annealing temperature; (b) obtain, from the spectrometer 75, a respective measured spectrum in response to the excitation light; (c) deconvolve the respective measured spectrum into a plurality of respective individual spectra, each of which corresponds to a respective one of the different molecules; and (d) output data corresponding to each of the respective individual spectra.

The FIG. 23A-D embodiment can be used to implement a method for detecting a presence or absence of a plurality of different molecules within a reaction container by performing the following steps: (a) illuminating contents of the reaction container using infrared light until a temperature within the reaction container reaches a denaturing temperature; (b) allowing the heated contents of the reaction container to cool until a temperature within the reaction container reaches an annealing temperature; (c) illuminating the contents of the reaction container with excitation light; (d) obtaining, while the contents of the reaction container are being illuminated with the excitation light, a respective measured spectrum of light that is being emitted by the contents of the reaction container; (e) deconvolving the respective measured spectrum into a plurality of respective individual spectra, each of which corresponds to a respective one of the different molecules; and (f) outputting data corresponding to each of the respective individual spectra. Steps (a) through (f) are repeated at least 10 times or at least 40 times.

### Example 16—Fast Optics-Based Thermocycling and RT-PCR Amplification

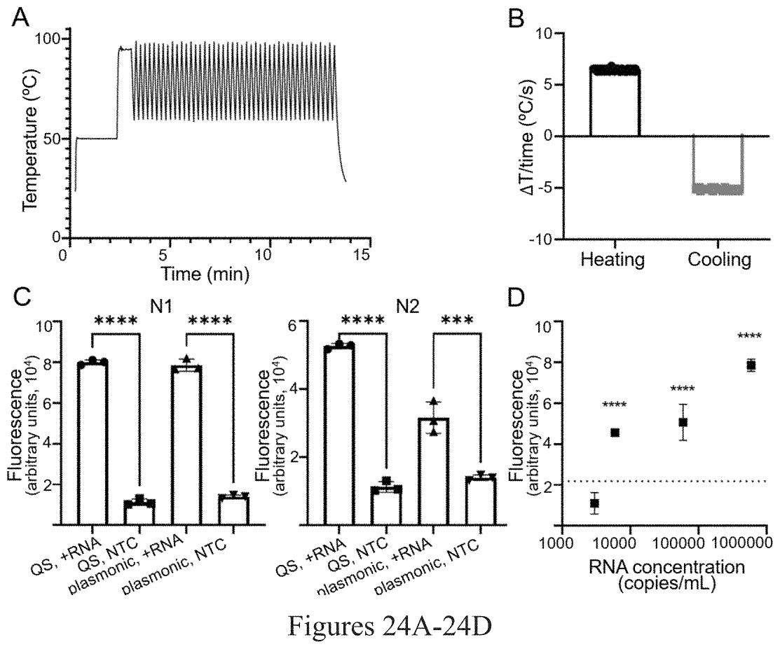

FIG. 24A-D depict achievement of fast, multiplexed, plasmonic RT-PCR using the embodiments described above in connection with FIGS. 23A-D. More specifically, FIG. 24A depicts well-controlled temperature sequences showing full RT-PCR in <15 minutes. Cycling parameters consisted of 2 minutes at 50° C., 10 seconds at 95° C., and 45 cycles at 60° C. for 2 seconds and 95° C. for 1 second.

FIG. 24B depicts consistent heating and cooling rates achieved throughout all 45 cycles, with an average heating rate of 6.48±0.14° C./s and an average cooling rate of −5.11±0.09° C./s (mean±SD, n=45). FIG. 24C depicts initial positive amplification results using a plasmonic RT-PCR system as measured by endpoint fluorescence on a plate reader. SARS-CoV-2 purified RNA in buffer was tested at a concentration of 5.9*10\hat{ }5 copies/mL. **** indicates adjusted p<0.0001, *** indicates p<0.0002, ns indicates ‘no significance’ as determined by one-way ANOVA followed by Tukey's multiple comparison tests.

FIG. 24D depicts initial photothermal amplification LoD data using purified RNA and fast amplification (<15 minutes). Threshold was determined by running three no template controls and taking the mean+10 standard deviations (dotted line). All concentrations were run in triplicate. Error bars show SD. Raw fluorescence for each concentration was compared to the NTC value via one-way ANOVA followed by Sidak's multiple comparisons test and determined to be statistically significant (**** indicates adjusted p<0.0001).

In these embodiments, fast thermocycling, and fast RT-PCR amplification of RNA targets using a streamlined workflow can be achieved without a RNA extraction step using optics-based heating. In one embodiment, a K-type wire thermocouple was inserted in the reaction vessel to measure temperature, and a proportional—integral—derivative (PID) control in a LabView-based thermocycling program was incorporated for precise temperature settings. This embodiment achieved initial temperatures for 2 minutes for reverse transcription and 10 seconds for denaturation of initial DNA template, followed by 45 cycles of rapid thermocycling, in 13 minutes total (FIG. 24A). Across the 45 cycles of thermocycling, the setup achieved a heating rate of 6.5±0.1° C./second and cooling rate of −5.1±0.1° C./second (FIG. 24B). Next, the inventors assessed whether RT-PCR was successful via fluorescence detection of amplified products from a template of purified SARS-CoV-2 RNA in buffer, without the need for a separate RNA extraction step. Via endpoint RT-PCR as assessed by a plate reader, for both N1 and N2 primer sets, this method produced significantly amplified products compared to a negative template control (NTC) using endpoint RT-PCR as assessed by a plate reader (FIG. 24C).

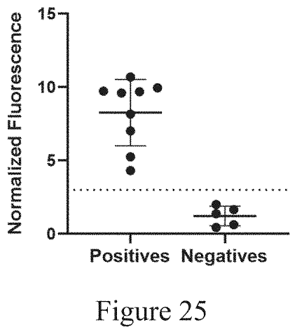

Human RNase P (RP) was amplified as an internal sample processing control for all reactions. This experiment produced a limit of detection (LoD) of 5.9×10³ copies/mL (FIG. 24D). Further, 14 human saliva specimens collected from symptomatic patients (using a setup with 2 LEDs) were tested using the exemplary system with a sensitivity of 100% and specificity of 100%, compared to laboratory-based PCR system (FIG. 25). While this set of experiments used a plate reader for end-point fluorescent measurements, this exemplary setup shows successful use of an optics-based method and system for rapid, extraction-free, plasmonic RT-PCR from SARS-CoV-2 RNA or other targets.

### Example 17—Integration of Multispectral Fluorescence Monitoring

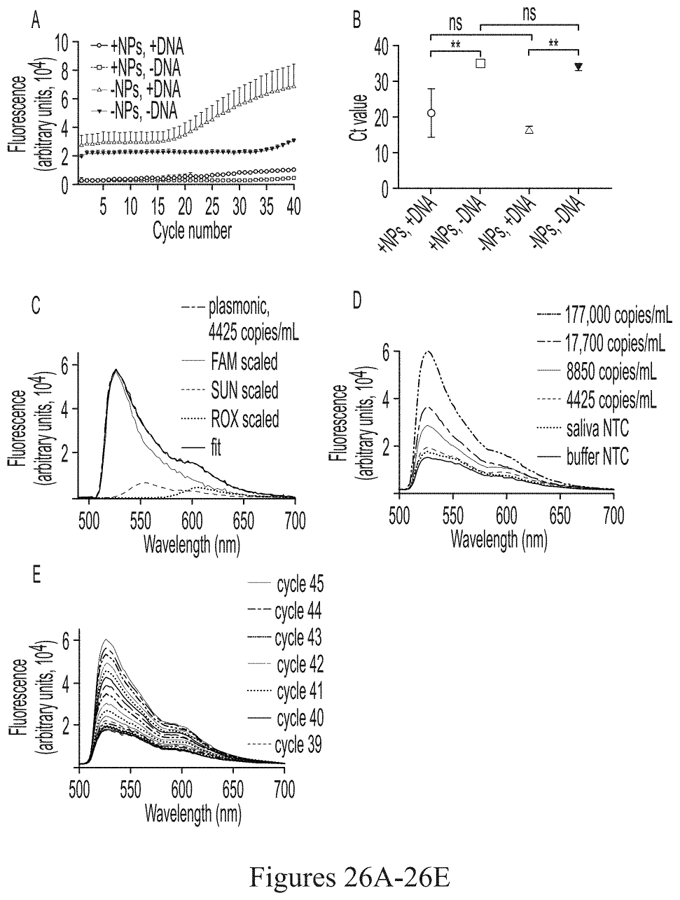

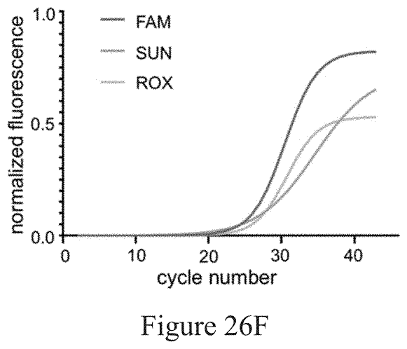

FIGS. 26A-F depict the integration of real-time fluorescent detection without removal of Au-NRs using the embodiments described above in connection with FIGS. 23A-D. This was done in the context of integration with on-board spectrometer and analytical characterization of integrated fast RT-PCR amplification of inactive SARS-CoV-2 virus spiked in patient saliva. More specifically, FIG. 26A depicts The addition of Au-NPs (OD18) to RT-PCR reactions quenches the raw fluorescent signal as shown by plotting the raw fluorescence as a function of cycle number from reactions on the Quant Studio instrument.

FIG. 26B depicts Ct values for data in (A) remain unaffected with and without nanoparticles (right panel). One-way ANOVA followed by Sidak's multiple comparisons test was performed, where ** indicates adjustedp<0.01, ns indicates no statistical significance. FIG. 26C depicts multi-spectral detection was achieved using a single excitation laser and a spectrometer, and multiple linear regression for spectral deconvolution. The actual and predicted fluorescent measurements showed extremely high concordance as fit by a multiple linear regression model in GraphPad Prism 9.

FIG. 26D depicts endpoint fluorescence detection in the presence of Au-NPs, across various initial concentrations of inactivated virus spiked in saliva, shows the ability for on-board fluorometry to detect different levels of amplicons. N1 amplification is detected via FAM, N2 via SUN, and RP via ROX. FIG. 26E depicts real-time amplification and detection as shown through both raw spectral curves increasing over time. And FIG. 26F plots deconvolved fluorescence values (for three individual targets/colors, fit to a sigmoidal curve) against cycle number in order to calculate Ct values.

In the FIGS. 23A-D embodiments, multispectral fluorescence detection was integrated into the infrared thermocycling setup to detect fluorescent signals generated by molecular probes in real time. For fluorescence excitation, a high-powered, single-color laser diode was used. For multi-color detection, a collimating lens, optical fiber, and a spectrometer was used. Microcontroller software was programmed to take measurements at the end of each cycle as described herein. Quenching of fluorescence of AuNRs has previously been described. Cheong et al., Fast detection of SARS-CoV-2 RNA via the integration of plasmonic thermocycling and fluorescence detection in a portable device, Nature Biomedical Engineering, volume 4, pages 1159-1167(2020).

Potential interference of AuNRs in the reaction vessel with fluorescence during a PCR process was examined. In a PCR reaction, despite some fluorescence quenching at a lowered optical density of AuNRs, fluorescence could still be monitored effectively over the duration of a full qPCR amplification process for amplifying spiked SARS-CoV-2 DNA plasmids in human saliva (FIG. 26A). Quantitatively, the Ct values calculated from fluorescence in the presence of AuNRs were unaffected compared to Ct values calculated in their absence (FIG. 26B).

The FIGS. 23A-D embodiments used a multispectral fluorescence detector setup in which the full wavelength spectra collected by the spectrometer 75 is separated into individual spectra for each of the relevant molecules (e.g., FAM, SUN, and ROX) using deconvolution. The deconvolution converts the measured spectrum that is obtained at the end of each cooling cycle into a linear combination of the spectra for each of the relevant individual molecules, and to find the proportionality coefficients. For example, when the relevant individual molecules are the FAM, SUN, and ROX dyes, the following equation can be used:

[Measured data]=A*[FAMspectrum]+B*[SUNspectrum]+C*[ROXspectrum]+D

where D is a constant offset. A standard algorithm (such as a Linear Least Squares Regression algorithm) is then used to find the best-fit coefficients for each of the cycles (e.g., at the end of each cooling cycle). In alternative embodiments, fluorescent dyes other than FAM, SUN, and ROX may be used (e.g., HEX, or another fluorescent dye having an excitation wavelength between 480 and 600 nm and an emission wavelength between 500 and 625 nm).

Based on amplification in the exemplary system and fluorescence measurements of individual targets, the deconvolution algorithm allowed for deconvolution into individual peaks and simultaneous detection of three nucleic-acid targets with three real-time probes tagged with FAM, SUN, and ROX dyes (FIG. 26C).

Using endpoint fluorescence detection, the ability of the setup, with integrated plasmonic thermocycling and multispectral fluorescence monitoring, to amplify and detect inactivated SARS-CoV-2 virus particles spiked into human saliva was tested. The N1 target, as indicated by FAM, as well as detection of RNase P, as indicated by ROX, were used as initial targets. Using virus concentrations ranging from ˜18,000 to ˜4,000 copies/mL in human saliva as the positive samples (within the range of SARS-CoV-2 virus exhibited in saliva of most patients), the fluorescence values of runs performed on positive samples were clearly distinguishable from those run on samples without templates (NTCs), with the endpoint fluorescent signals across the spectra being proportional to the amount of starting virus in the sample (FIG. 26D). Zhao et al., Viral dynamics of SARS-CoV-2 in saliva from infected patients, J Infect. 2020 Sep; 81(3); Wyllie et al., Saliva or Nasopharyngeal Swab Specimens for Detection of SARS-CoV-2, N Engl J Med 2020; 383:1283-1286.

Real-time fluorescence monitoring was enabled by modifying the software to trigger the laser and spectrometer after each cycle. Monitoring the fluorescence of FAM and ROX in human saliva showed the amplitude of the spectra increasing with each cycle over time, indicating performance of real-time RT-PCR (FIG. 26E). Plotting this data against cycle number confirmed the ability to obtain amplification curves and cycle threshold (Ct) values, for three fluorophores (i.e., targets) simultaneously, in real time (FIG. 26F).

### Example 18—Testing With Spiked Inactivated SARS-CoV-2 Viruses in Human Saliva

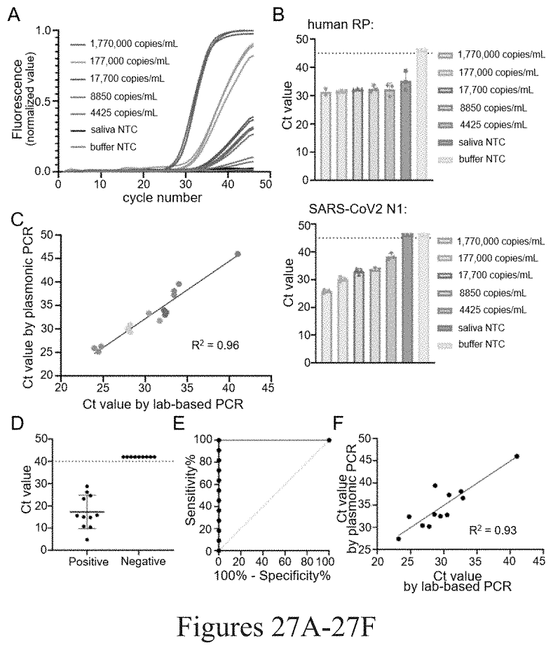

Inactivated SARS-CoV-2 virus spiked into human saliva on the exemplary real-time plasmonic RT-PCR instrument was tested. This test successfully detected virus concentrations as low as 4425 copies/mL (FIG. 27A) (below the viral load in saliva of most COVID-19 human subjects (Wyllie et al., Saliva or Nasopharyngeal Swab Specimens for Detection of SARS-CoV-2, N Engl J Med 2020; 383:1283-1286)). Ct values (used as a control) for human RP were similar across different concentrations of spiked SARS-CoV-2, which was different from NTC (FIG. 27B, top). By comparison, log-fold differences in copy numbers produced smaller Ct values as virus concentrations increased, as expected (FIG. 27B, bottom). Further analyzing the quantitative capability of plasmonic RT-qPCR, the Ct values (for viral N1 detection) from each concentration were compared against those samples run on a laboratory-based PCR device. The Ct values for the two approaches exhibited a correlation coefficient of 0.96, further demonstrating the utility of the FIGS. 23A-D embodiments in quantitative analysis (FIG. 27C).

The FIGS. 23A-D embodiments were used to perform testing with SARS-CoV-2 human clinical specimens. 21 human saliva specimens (11 positives, 10 negatives) were tested with plasmonic thermocycling and real-time fluorescence monitoring. The runs took 21.0±1.1 minutes. The setup yielded a sensitivity of 100% and specificity of 100% (FIG. 27D), and an area under the curve (AUC) of 1.0 (FIG. 27E) when compared to a reference of laboratory-based PCR. The quantitative capability of the setup was evaluated. The Ct values obtained exhibited a correlation coefficient of 0.93 compared to the Ct values of the same samples run on a laboratory-based PCR instrument (FIG. 27F). Past studies have placed Ct values into bins (for example, 5 cycles per bin or cutoff threshold values) to present different viral loads that correlated with severity of COVID-19 disease.

Finally, for the COVID-19 application, the inventors noted that the extraction-free processing of human saliva specimens as input into plasmonic RT-qPCR could potentially result in a simple workflow when considered more broadly from sample collection to result.

Plasmonic RT-qPCR has multiple advantages for diagnosis of COVID-19 and other infectious diseases compared to previous devices and methods. First, since fluorescence measurements do not require the AuNRs to be removed from the vessel, concerns for biosafety for the user at a POC setting are reduced. Second, early real-time monitoring could give rapid results for strongly positive specimens with low Ct values. Third, Ct values are widely analyzed in clinical diagnostics, while not yet conclusive for COVID-19. There is strongly suggestive evidence that Ct values correlate with severity. For example, patients with severe COVID-19 tend to have a high viral load and a long virus-shedding period, pointing to utility of measuring viral load as an indicator of disease severity and prognosis. Fourth, the ability to monitor and detect more than one viral target in addition to a human control target is of increasing relevance for detecting SARS-CoV-2 variants. Fifth, a demonstration of integrated sample-to-result workflow further highlights the potential of plasmonic thermocycling for real clinical utility. Moreover, because of potential shortage of RNA extraction reagents, extraction-free methods that avoid purification could be advantageous. The apparatus and methods described herein demonstrate that use of plasmonic RT-PCR on clinical specimens from sample collection to result, and provide significant workflow advantages (e.g., use of extraction-free saliva analysis). Overall, the quantitation and multiplexing capabilities of the FIGS. 23A-D embodiments facilitate PCR capabilities using an all-optics approach offered in a POC format with rapid thermocycling.

### Example 19—Design of Instrument for Plasmonic Thermocycling

The FIGS. 23A-D embodiments use both plasmonic thermocycling and multispectral fluorometer, both of which can operate on a same reaction vessel with no moving components or steps. The plasmonic thermocycling preferably provides: (1) consistent heating via Au-NRs; (2) real-time fluorescent detection; and (3) compatibility with a simple sample cartridge. In one embodiment that was implemented and tested, a hexagonal 3D printed hub was designed to contain all optical components concentrically surrounding the PCR tube. Three infrared LEDs were positioned underneath a lens (ThorLabs) and on top of a custom machined heat sink and fan (Digi-Key) to prevent overheating. An additional 12V fan (Digi-Key) was positioned near the PCR tube and used to cool the sample. The fluorometer (laser and spectrometer setup, FIG. 23C) was positioned in the remaining sides of the hub. The fluorometer used a 488 nm laser diode as an excitation source. An airflow chamber was cut out of the 3D printed hub to ensure airflow to the PCR tube.

For initial experiments (FIGS. 24A-24D), K-type thermocouples (Evolution Sensors) were used to measure temperature and control thermal cycling in a closed-loop form using a custom Lab View software with integrated PID control. For downstream experiments (FIGS. 26A-26F, FIGS. 27A-27F), a corollary LabView program was written to enable input of LED intensity and time sequences for open-loop control of the optics.

### Example 20—Testing of Amplification Using Plasmonic RT-PCR

Functionalized silica-coated gold nanorods (Au-NRs) were purchased from Nanopartz. Au-NRs had an SPR peak ˜850 nm (slight variation between batches) and an aspect ratio ˜4.5 nm. A 10 nm silica coating was used to prevent adsorption of proteins during the PCR reaction.

PCR reactions used 10 uL 2× PrimeScript III (TaKaRa, 1× final concentration), 0.57 uL Au-NRs (Nanopartz, final concentration OD of 2), 500 nM forward and reverse primers (IDT) and 125 nM probes (IDT). Spiked RNA (BEI) in 1× TE buffer (10 mM Tris-HCl, 1 mM EDTA) or spiked inactivated virus (BEI) in 1:1 mixtures of donor saliva (Innovative Research) and 1× TE buffer were used to bring the reaction volume up to 20 uL. No template controls (NTC) were tested with the same mix and conditions but without SARS-CoV-2 RNA (FIGS. 24A-24D) or inactivated virus (FIGS. 26A-26D, FIGS. 27A-27F). Buffer NTCs indicate the use of TE buffer only, and saliva NTCs indicate the use of donor saliva mixed 1:1 with TE buffer as described above. A volume of 15 uL of ChillOut Liquid Wax (Bio-Rad) was used to prevent evaporation during thermal cycling.

Closed-loop thermal cycling conditions for experiments using purified RNA spiked in buffer (FIGS. 24A-24D) were programmed in LabView as follows: 2 minutes at 50° C., 10 seconds at 95° C., followed by 45 cycles at 60° C. for 2 seconds and 95° C. for 1 second.

Thermocycling conditions for experiments that used inactivated virus spiked in donor saliva (FIGS. 26A-26F, FIGS. 27A-27F) were initially programmed in LabView using a closed-loop control, and the output was converted to an open loop format for testing (except FIG. 27E and 27F which used a closed loop configuration). The closed-loop cycling parameters varied only slightly between experiments. The reverse transcription step was 50° C. for 2 min or 5 min, followed by initial denaturation for 10 seconds at 95° C. Next, the reaction cycled 45 times between 58 to 60° C. for 2 to 8s and 91 to 97° C. for 1 second. Samples were considered positive on the prototype if both N1 and RP were detected (Ct<46). As commonly done for diagnostic tests, samples for which human RP failed to amplify were considered indeterminate.

Example 21—Methods of Detection of SARS-CoV-2 RNA from Human Saliva Samples

De-identified clinical specimens were obtained under a protocol approved by the Columbia University Medical Center IRB (AAAT0100). Saliva specimens were obtained from Mirimus Foundation and SUNY Downstate and stored at 4° C. upon receipt. Samples tested in FIG. 24D were heat inactivated prior to receipt by heating at 95° C. for 5 minutes. Samples tested in FIGS. 27A-27F were from patients suspected of being infected with SARS-CoV-2, and not heat inactivated. Samples were diluted 1:1 in 1× TE buffer and added to PCR reaction mix as described above. Although all clinical testing occurred within 2 weeks of receiving the samples, in light of possible sample degradation during transportation and storage, the status of the specimens were re-confirmed by running them in duplicates on laboratory-based qPCR using a ThermoFisher QuantStudio instrument, which acted as a laboratory-based PCR reference.

The results were fully concordant with the reference results from Mirimus Foundation, with the exception of one specimen which tested positive from the vendor but which tested negative (in duplicate) on a Quantstudio instrument, pointing to possible RNA degradation during transportation and storage. A sample was considered positive if N1 and/or RP was detected, negative if N1 was not detected and RP was detected, and indeterminate if neither N1 nor RP was detected. For the Rover instrument, all clinical samples were tested once if positive or negative, and twice if indeterminate. If an indeterminate sample tested either positive or negative upon repeating the run, that result was used. A sample was considered negative if RP was detected but N1 was not detected.

### Example 22—Fluorescence Measurements and Ct Calculations

Fluorescent measurements (FIGS. 24A-24D) were made using a 384-well plate and using a plate reader (Biotek) for 5-FAM and ROX dyes (N1 and RP, respectively). Fluorescent measurements on the prototype (FIGS. 26A-26F, FIGS. 27A-27F) were made as follows: raw fluorescence spectra were collected by the spectrometer at the end of annealing/extension step during each cycle. These spectra were analyzed using least squares regression, based on ideal peaks determined experimentally by measuring the fluorescent spectra for amplicons containing PCR product from a single fluorophore. Next, each component signal was plotted against cycle number. The baseline was pre-selected to be cycles 3-25, and the threshold was calculated as the mean +10 standard deviations above the baseline. The Ct value was calculated to be the interpolated cycle at which the fluorescent signal crossed the calculated threshold value. (Samples that did not produce a sigmoidal curve were excluded from analysis.)

All statistics, including one-way ANOVA followed by Sidak's multiple comparison tests, and one-way ANOVA followed by Tukey's multiple comparison tests, were performed using GraphPad Prism 9 software.

While the aspects described herein have been disclosed with reference to certain embodiments, numerous modifications, alterations, and changes to the described aspects are possible without departing from the sphere and scope of the present invention, as defined in the appended claims. Accordingly, it is intended that the present invention not be limited to the described aspects, but that it has the full scope defined by the language of the following claims, and equivalents thereof.

What is claimed is:

1. A method of detecting a nucleic acid in a single reaction chamber, comprising:
(a) obtaining a patient specimen suspected of comprising a first nucleic acid;
(b) forming a crude lysate from the patient specimen;
(c) forming a reaction mixture by combining the crude lysate with an infrared-absorbing material, a detecting nucleic acid, and at least one reporter molecule in the single reaction chamber;
(d) heating the reaction mixture to at least 35° C. by irradiating the reaction mixture with infrared light; and
(e) detecting a presence of the at least one reporter molecule, wherein the presence of the at least one reporter molecule indicates the patient specimen contains the first nucleic acid, and wherein steps (b) through (d) occur in the single reaction chamber.

2. The method of claim 1, wherein the at least one reporter molecule comprises at least two reporter molecules.

3. The method of claim 1, wherein the infrared-absorbing material comprises gold nanoparticles.

4. The method of claim 1, wherein the first nucleic acid is amplified using one of polymerase chain reaction (PCR) or isothermal amplification.

5. The method of claim 4, wherein the isothermal amplification comprises loop-mediated isothermal amplification (LAMP).

6. The method of claim 1, wherein the heating of the reaction mixture denatures the first nucleic acid at a denaturing temperature.

7. The method of claim 6, further comprising cooling the reaction mixture to an annealing temperature after step (d), and allowing the detecting nucleic acid to anneal to the first nucleic acid, forming an annealed nucleic acid.

8. The method of claim 7, wherein a temperature within the reaction chamber cycles between a denaturing temperature and an annealing temperature at least 10 times.

9. The method of claim 7, further comprising adding nucleotides to the reaction mixture, and allowing extension of the annealed nucleic acid with the nucleotides.

10. The method of claim 1, wherein the first nucleic acid is ribonucleic acid (RNA), further comprising reverse transcribing the RNA prior to the heating of the reaction mixture.

11. A method for detecting a presence or absence of a plurality of different molecules within a reaction container comprising:
(a) illuminating contents of the reaction container using infrared light until a temperature within the reaction container reaches a denaturing temperature;
(b) allowing the heated contents of the reaction container to cool until a temperature within the reaction container reaches an annealing temperature;
(c) illuminating the contents of the reaction container with excitation light;
(d) obtaining, while the contents of the reaction container are being illuminated with the excitation light, a respective measured spectrum of light that is being emitted by the contents of the reaction container;
(e) deconvolving the respective measured spectrum into a plurality of respective individual spectra, each of which corresponds to a respective one of the different molecules;
(f) outputting data corresponding to each of the respective individual spectra; and
(g) repeating steps (a) through (f) at least 10 times.

12. The method of claim 11, wherein step (g) comprises repeating steps (a) through (f) at least 40 times.

13. The method of claim 11, wherein the plurality of different molecules comprises at least three different molecules.

14. The method of claim 11, wherein the plurality of different molecules comprises at least two molecules selected from the group consisting of FAM, SUN, HEX, and ROX.

15. The method of claim 11, wherein each of the plurality of different molecules comprises a fluorescent dye having an excitation wavelength between 480 and 600 nm and an emission wavelength between 500 and 625 nm.

16. The method of claim 11, wherein the reaction container contains gold nanoparticles dispersed in a liquid.

17. An apparatus for detecting a presence or absence of a plurality of different molecules within a reaction container (40), the apparatus comprising:
an infrared light source (50) aimed to illuminate contents of the reaction container;
an excitation light source (95) positioned to illuminate contents of the reaction container; and
a spectrometer (75) positioned to detect emission light emanating from the reaction container during times when the excitation light source is illuminating the contents of the reaction container; and
a controller programmed to, for each of N cycles,
(a) control the infrared light source so that a temperature within the reaction container cycles between a denaturing temperature and an annealing temperature,
(b) obtain, from the spectrometer, a respective measured spectrum in response to the excitation light,
(c) deconvolve the respective measured spectrum into a plurality of respective individual spectra, each of which corresponds to a respective one of the different molecules, and
(d) output data corresponding to each of the respective individual spectra, wherein N is an integer greater than or equal to 10.

18. The apparatus of claim 17, wherein N is greater than or equal to 40.

19. The apparatus of claim 17, wherein each of the plurality of different molecules comprises a fluorescent dye having an excitation wavelength between 480 and 600 nm and an emission wavelength between 500 and 625 nm.

20. The apparatus of claim 17, further comprising the reaction container (40), wherein the reaction container contains gold nanoparticles dispersed in a liquid.
